(19) United States
(12) Patent Application Publication
(10) Pub. No.: US 2024/0132511 A1
(43) Pub. Date: Apr. 25, 2024

(54) TREATMENTS OF PRADER-WILLI SYNDROME

(71) Applicant: Neuren Pharmaceuticals Limited, Auckland (NZ)

(72) Inventors: Lawrence Irwin GLASS, Flintstone (NZ); Patricia COGRAM, Oxford (GB)

(21) Appl. No.: 18/546,234

(22) PCT Filed: Feb. 11, 2022
(86) PCT No.: PCT/NZ2022/050017

§ 371 Date: Aug. 11, 2023

Related U.S. Application Data
(60) Provisional application No. 63/148,962, filed on Feb. 12, 2021.

(51) Int. Cl. C07D 487/04; A61K 45/06; A61P 3/04; A61P 43/00

(52) U.S. Cl. CPC C07D 487/04; A61K 45/06; A61P 3/04; A61P 43/00

(57) ABSTRACT

The present disclosure generally relates to methods of and uses for treating Prader-Willi Syndrome (PWS) in a subject, comprising administering to the subject a therapeutically effective amount of a compound of Formulas I, II, III or IV, or a pharmaceutically acceptable salt, hydrate, stereoisomer, or prodrug thereof, or for use in the manufacture of a medicament as described herein.

### FIELD

The present disclosure generally relates to the field of pharmaceutical treatments of Prader-Willi Syndrome. More specifically, the present disclosure relates to the use of bicyclic compounds in the treatment of Prader-Willi Syndrome.

### BACKGROUND

Prader-Willi Syndrome (PWS) is a rare genetic condition caused by loss of function in the 15q11-q13 region on the paternal copy of chromosome 15. Approximately 70% of cases are caused by deletion of a segment of the paternal copy of chromosome 15 and approximately 25% by duplication of the maternal copy of chromosome 15 (uniparental disomy (UPD)). PWS has an estimated birth incidence of between 1:10,000 and 1:30,000 and an estimated prevalence of approximately 10,000 to 20,000 living individuals in the United States. PWS affects both males and females and can affect individuals of any ethnic or racial background (Bohonowych et al. 2019).

PWS is characterised by early failure to thrive followed by the onset of excessive appetite (hyperphagia), which frequently results in obesity, type 2 diabetes, and metabolic syndrome. Additional characteristics include multiple endocrine abnormalities, hypotonia, hypogonadism, sleep disturbances, a challenging neurobehavioral phenotype, self-injurious behaviour, mild to moderate intellectual and learning disabilities, and distinctive facial features (Bohonowych et al. 2019).

### SUMMARY

Current treatments for Prader-Willi Syndrome are limited. Human growth hormone can improve muscle tone and reduces body fat. Testosterone can be used to treat males, and estrogen and progesterone can be used to treat females to reduce the risk of osteoporosis. Sleep apnea and other sleep problems can be treated to improve behavioral issues. Behavioral therapy can help controlling behavioural problems. Some medications could be provided. Physiotherapy, speech therapy, developmental therapy can help improve social and inter-personal skills. Current treatments for PWS are limited and, to date, focus on the treatment of endocrine abnormalities with growth hormone therapy (Bohonowych et al. 2019).

Accordingly, there remains a need for alternative and effective therapies for the treatment of Prader-Willi Syndrome and/or symptoms associated with Prader-Willi Syndrome.

The subject matter of the present disclosure is predicated in part on the surprising discovery that compounds of Formula I, or a pharmaceutically acceptable salt, hydrate, stereoisomer, or prodrug thereof, may treat Prader-Willi Syndrome and/or symptoms associated with Prader-Willi Syndrome.

Accordingly, in a first aspect, there is provided a method of treating Prader-Willi Syndrome in a subject, comprising administering to the subject a therapeutically effective amount of a compound of Formula I, or a pharmaceutically acceptable salt, hydrate, stereoisomer, or prodrug thereof:

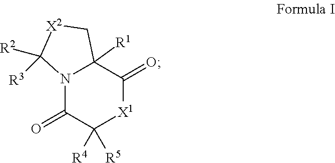

wherein X¹ is selected from the group consisting of NR′, O, and S; X² is selected from the group consisting of CH₂, NR′, O, and S; R¹, R², R³, R⁴, and R⁵ are independently selected from the group consisting of hydrogen, halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, alkynyl, 3-10-membered carbocycle, and 3-10-membered heterocycle, wherein each alkyl, heteroalkyl, alkenyl, alkynyl, 3-10-membered carbocycle, and 3-10-membered heterocycle is unsubstituted or substituted with one or more substituents selected from the group consisting of halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, and alkynyl; wherein each R′ is independently selected from the group consisting of hydrogen, alkyl, heteroalkyl, alkenyl, alkynyl, 3-10-membered carbocycle, and 3-10-membered heterocycle; or R⁴ and R⁵ taken together are —CH₂—(CH₂)ₙ—CH₂— where n is an integer from 0-6; or R² and R³ taken together are —CH₂—(CH₂)ₙ—CH₂— where n is an integer from 0-6; with the proviso that when R¹ is CH³, R² is hydrogen, R³ is hydrogen, and R⁴ is hydrogen, then R⁵ is not benzyl; and when R¹ is hydrogen, then at least one of R² and R³ is not hydrogen.

In another aspect, there is provided use of a compound of Formula I, or a pharmaceutically acceptable salt, hydrate, stereoisomer, or prodrug thereof:

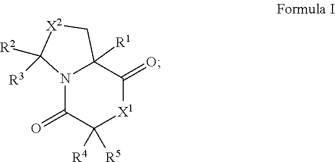

wherein X¹ is selected from the group consisting of NR′, O, and S; X² is selected from the group consisting of CH₂, NR′, O, and S; R¹, R², R³, R⁴, and R⁵ are independently selected from the group consisting of hydrogen, halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, alkynyl, 3-10-membered carbocycle, and 3-10-membered heterocycle, wherein each alkyl, heteroalkyl, alkenyl, alkynyl, 3-10-membered carbocycle, and 3-10-membered heterocycle is unsubstituted or substituted with one or more substituents selected from the group consisting of halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, and alkynyl; wherein each R′ is independently selected from the group consisting of hydrogen, alkyl, heteroalkyl, alkenyl, alkynyl, 3-10-membered carbocycle, and 3-10-membered heterocycle; or R⁴ and R⁵ taken together are —CH₂—(CH₂)ₙ—CH₂— where n is an integer from 0-6; or R² and R³ taken together are —CH₂—(CH₂)ₙ—CH₂— where n is an integer from 0-6; with the proviso that when R¹ is CH₃, R² is hydrogen, R³ is hydrogen, and R⁴ is hydrogen, then R⁵ is not benzyl; and when R¹ is hydrogen, then at least one of R² and R³ is not hydrogen; in the manufacture of a medicament for the treatment of Prader-Willi Syndrome.

In a further aspect, there is provided a compound of Formula I, or a pharmaceutically acceptable salt, hydrate, stereoisomer, or prodrug thereof:

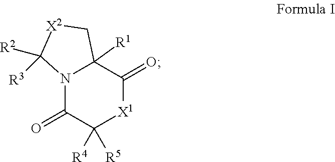

wherein X¹ is selected from the group consisting of NR′, O, and S; X² is selected from the group consisting of CH₂, NR′, O, and S; R¹, R², R³, R⁴, and R⁵ are independently selected from the group consisting of hydrogen, halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, alkynyl, 3-10-membered carbocycle, and 3-10-membered heterocycle, wherein each alkyl, heteroalkyl, alkenyl, alkynyl, 3-10-membered carbocycle, and 3-10-membered heterocycle is unsubstituted or substituted with one or more substituents selected from the group consisting of halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, and alkynyl; wherein each R′ is independently selected from the group consisting of hydrogen, alkyl, heteroalkyl, alkenyl, alkynyl, 3-10-membered carbocycle, and 3-10-membered heterocycle; or R⁴ and R⁵ taken together are —CH₂—(CH₂)ₙ—CH₂— where n is an integer from 0-6; or R² and R³ taken together are —CH₂—(CH₂)ₙ—CH₂— where n is an integer from 0-6; with the proviso that when R¹ is CH₃, R² is hydrogen, R³ is hydrogen, and R⁴ is hydrogen, then R⁵ is not benzyl; and when R¹ is hydrogen, then at least one of R² and R³ is not hydrogen; for use in treating Prader-Willi Syndrome.

It will be appreciated that other aspects, embodiments, and examples of the compounds, pharmaceutical compositions, methods, or uses, are further described herein.

### BRIEF DESCRIPTION OF THE DRAWINGS

Whilst it will be appreciated that a variety of embodiments of the disclosure may be utilised, in the following, a number of examples of the disclosure with reference to the following drawings are described:

FIG. 1 shows the chemical structure of cG-2-AllylP (also known as “NNZ2591”).

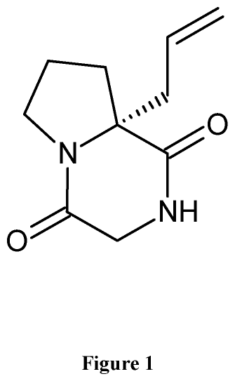

FIG. 2 shows a graph of the results of an open field study of the effects of cG-2-AllylP or vehicle in Magel2-null mice on hypoactivity (distance travelled) compared to wild-type mice.

FIG. 3 shows a graph of the results of an open field study of the effects of cG-2-AllylP or vehicle in Magel2-null mice on hypoactivity (time spent active) compared to wild-type mice.

FIG. 4 shows a graph of the results of studies of the effects of cG-2-AllylP or vehicle in Magel2-null mice on daily living (nesting) compared to wild-type mice.

FIG. 5 shows a graph of the results of studies of the effects of cG-2-AllylP or vehicle in Magel2-null mice on social preference compared to wild-type mice.

FIG. 6 shows a graph of the results of studies of the effects of cG-2-AllylP or vehicle in Magel2-null mice on social interaction compared to wild-type mice.

FIG. 7 shows a graph of the results of studies of the effects of cG-2-AllylP or vehicle in Magel2-null mice on cognition (novel object recognition) compared to wild-type mice.

FIG. 8 shows graphs of the results of studies of the effects of cG-2-AllylP or vehicle in Magel2-null mice on anxiety (elevated plus maze: time spent in open arms) compared to wild-type mice.

FIG. 9 shows a graph of the results of studies of the effects of cG-2-AllylP or vehicle in Magel2-null mice on fat mass compared to wild-type mice.

FIG. 10 shows a graph of the results of studies of the effects of cG-2-AllylP or vehicle in Magel2-null mice on circulating IGF-1 levels compared to wild-type mice.

### DETAILED DESCRIPTION

### General Definitions

Unless specifically defined otherwise, all technical and scientific terms used herein shall be taken to have the same meaning as commonly understood by one of ordinary skill in the art (e.g., chemistry, biochemistry, medicinal chemistry, microbiology and the like).

As used herein, the term “and/or”, e.g., “X and/or Y”, shall be understood to mean either “X and Y” or “X or Y” and shall be taken to provide explicit support for both meanings or for either meaning, e.g., A and/or B includes the options i) A, ii) B, or iii) A and B.

As used herein, the term “about”, unless stated to the contrary, refers to +/−20%, typically +/−10%, typically +/−5%, of the designated value.

As used herein, the terms “a”, “an”, and “the” include both singular and plural aspects, unless the context clearly indicates otherwise.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

Throughout the present specification, various aspects and components of the invention can be presented in a range format. The range format is included for convenience and should not be interpreted as an inflexible limitation on the scope of the invention. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 3, from 1 to 4, from 1 to 5, from 2 to 4, from 2 to 5, from 3 to 5 etc., as well as individual and partial numbers within the recited range, for example, 1, 2, 3, 4, 5, 5.5 and 6, unless where integers are required or implicit from context. This applies regardless of the breadth of the disclosed range. Where specific values are required, these will be indicated in the specification.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The phrase “consisting of” means the enumerated elements and no others.

The phrase “consisting essentially of” means the enumerated elements and their equivalents.

It will be clearly understood that, although a number of prior art publications are referred to herein, this reference does not constitute an admission that any of these documents form part of the common general knowledge in the art in the United States, Australia, or in any other country.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will prevail. In addition, the materials, methods, and examples are illustrative only and not intended to be limiting.

As used herein, the term “subject” refers to any organism susceptible to a disease or condition. For example, the subject can be a mammal, primate, livestock (e.g., sheep, cow, horse, pig), companion animal (e.g., dog, cat), or laboratory animal (e.g., mouse, rabbit, rat, guinea pig, hamster). In one embodiment, the subject is a mammal. In one example, the subject is human. In one embodiment, the disease or condition is Prader-Willi Syndrome.

As used herein, the term “treating” includes improving, alleviating, reducing, or eliminating one or more symptoms associated with a specific disorder or condition. In one embodiment, the disorder or condition is Prader-Willi Syndrome. For example, as used herein, the phrase “treating Prader-Willi Syndrome” includes improving, alleviating, reducing, or eliminating one or more symptoms associated with Prader-Willi Syndrome.

As used herein, the term “prevention” includes prophylaxis of the specific disorder or condition, or one or more symptoms associated with a specific disorder or condition. In one embodiment, the one or more symptoms is one or more symptoms associated with Prader-Willi Syndrome. For example, as used herein, the phrase “preventing Prader-Willi Syndrome” refers to preventing the onset or duration of one or more symptoms associated with Prader-Willi Syndrome. In some embodiments, the phrase “preventing Prader-Willi Syndrome” refers to slowing or halting the progression of one or more symptoms of Prader-Willi Syndrome. Prevention may be absolute (such that there is no presentation of a particular symptom) or may be effective only in some individuals, to some extent, or for a limited amount of time.

The present disclosure relates to compounds of Formula I and pharmaceutically acceptable salts, hydrates, stereoisomers, and prodrugs thereof.

Salts may be formed in the case of embodiments of the compound of Formula I which contain a suitable acidic or basic group. Suitable salts of the compound of Formula I include those formed with organic or inorganic acids or bases.

As used herein, the phrase “pharmaceutically acceptable salt” or a like term refers to pharmaceutically acceptable organic or inorganic salts. It will be appreciated that any reference to “salt” herein can include “pharmaceutically acceptable salts”. Exemplary acid addition salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. Exemplary base addition salts include, but are not limited to, ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion. It will also be appreciated that non-pharmaceutically acceptable salts also fall within the scope of the present disclosure since these may be useful as intermediates in the preparation of pharmaceutically acceptable salts or may be useful during storage or transport.

Those skilled in the art of organic and/or medicinal chemistry will appreciate that many organic compounds can form complexes with solvents in which they are reacted or from which they are precipitated or crystallized. These complexes are known as “solvates”. For example, a complex with water is known as a “hydrate”. As used herein, the phrase “pharmaceutically acceptable solvate” or “solvate” refer to an association of one or more solvent molecules and a compound of the present disclosure. Examples of solvents that form pharmaceutically acceptable solvates include, but are not limited to, water, isopropanol, ethanol, methanol, DMSO, ethyl acetate, acetic acid, and ethanolamine. It will be understood that the present disclosure encompasses solvated forms, including hydrates, of the compounds of Formula I and salts thereof.

The compounds of the present disclosure may contain chiral (asymmetric) centers or the molecule as a whole may be chiral. The individual stereoisomers (enantiomers and diastereoisomers) and mixtures of these are within the scope of the present disclosure.

As used herein, the term “stereoisomer” refers to compounds having the same molecular formula and sequence of bonded atoms (i.e., atom connectivity), though differ in the three-dimensional orientations of their atoms in space. As used herein, the term “enantiomers” refers to two compounds that are stereoisomers in that they are non-superimposable mirror images of one another. Relevant stereocenters may be denoted with (R)- or (S)-configuration.

Those skilled in the art of organic and/or medicinal chemistry will appreciate that the compounds of Formula I and salts thereof may be present in an amorphous form or in a crystalline form. It will be understood that the present disclosure encompasses all forms and polymorphs of the compounds of Formula I and salts thereof.

As used herein, the term “protecting group” has the meaning conventionally associated with it in organic synthesis/medicinal chemistry, i.e., a chemical group that selectively blocks one or more reactive sites in a multifunctional compound such that a chemical reaction can be carried out selectively on another unprotected reactive site and such that the group can readily be removed after the selective reaction is complete.

As would be understood by the person skilled in the art, a compound of Formula I, or any salt, solvate or stereoisomer thereof would be administered in a therapeutically effective amount. The term “therapeutically effective amount”, as used herein, refers to a compound being administered in an amount sufficient to alleviate or prevent to some extent one or more of the symptoms of the disorder or condition being treated. The result can be the reduction and/or alleviation of the signs, symptoms, or causes of a disease or condition, or any other desired alteration of a biological system. In one embodiment, the term “therapeutically effective amount” refers to a compound of Formula I, or any salt thereof, being administered in an amount sufficient to treat and/or prevent one or more symptoms associated with Prader-Willi Syndrome so as to provide a therapeutic outcome.

Implicit hydrogen atoms (such as the hydrogen atoms present on the pyrrole ring, etc.) are omitted from the formulae for clarity, but would be understood by the skilled person to be present.

As used herein, the term “halogen” means fluorine, chlorine, bromine, or iodine.

As used herein, the term “alkyl” encompasses both straight-chain (i.e., linear) and branched-chain hydrocarbon groups. Examples of alkyl groups include, but are not limited to, methyl, ethyl, n-propyl, iso-propyl, n-butyl, t-butyl, i-butyl, sec-butyl, pentyl, and hexyl groups. In one example, the alkyl group is of one to six carbon atoms (i.e. C₁₋₆alkyl).

As used herein, the term “haloalkyl” refers to an “alkyl” group substituted with one or more “halogen” groups according to any examples thereof as separately described above or herein.

As used herein, the term “alkenyl” refers to both straight and branched chain unsaturated hydrocarbon groups with at least one carbon-carbon double bond. Examples of alkenyl groups include, but are not limited to, ethenyl, propenyl, butenyl, pentenyl, and hexenyl groups. In one example, the alkenyl group is of two to six carbon atoms (i.e. C₂₋₆alkenyl).

As used herein, the term “haloalkenyl” refers to an “alkenyl” group substituted with one or more “halogen” groups according to any examples thereof as separately described above or herein.

As used herein, the term “alkynyl” refers to both straight and branched chain unsaturated hydrocarbon groups with at least one carbon-carbon triple bond. Examples of alkynyl groups include, but are not limited to, ethynyl, propynyl, butynyl, pentynyl, and hexynyl groups. In one example, the alkynyl group is of two to six carbon atoms (i.e. C₂₋₆alkynyl).

As used herein, the term “carbocyclyl”, “carbocycle”, “carbocyclic”, or like term, refers to an aromatic or non-aromatic cyclic group of carbon atoms. A carbocyclyl group may, for example, be monocyclic or polycyclic (i.e. bicyclic, tricyclic). A polycyclic carbocyclyl group may contain fused rings. In one example, the carbocyclyl group is of three to ten carbon atoms (i.e. C₃₋₁₀carbocyclyl). In one example, the carbocyclyl group is of three to seven carbon atoms (i.e. C₃₋₇carbocyclyl). Examples of monocyclic non-aromatic carbocyclyl groups include cyclopropyl, cyclobutyl, cyclopentyl, cyclopentenyl, cyclohexyl, cyclohexenyl, cycloheptyl, and cyclooctyl groups. Examples of monocyclic saturated carbocyclyl groups include cyclopropyl, cyclobutyl, cyclopentyl, cyclohexyl, cycloheptyl, and cyclooctyl groups. Aromatic carbocyclyl groups include phenyl and napthalenyl.

As used herein, the term “heterocyclyl” refers to an aromatic or non-aromatic cyclic group which is analogous to a carbocyclic group, but in which from one to three of the carbon atoms is/are replaced by one or more heteroatoms independently selected from nitrogen, oxygen, or sulfur. A heterocyclyl group may, for example, be monocyclic or polycyclic (e.g. bicyclic). A polycyclic heterocyclyl may for example contain fused rings. In a bicyclic heterocyclyl group there may be one or more heteroatoms in each ring, or heteroatoms only in one of the rings. A heteroatom may be N, O, or S. Heterocyclyl groups containing a suitable nitrogen atom include the corresponding N-oxides. In one example, the heterocyclyl group is of three to ten atoms (i.e. 3-10-membered heterocyclyl). In one example, the heterocyclyl group is of three to seven atoms (i.e. 3-7-membered heterocyclyl). Examples of monocyclic non-aromatic heterocyclyl groups include aziridinyl, azetidinyl, pyrrolidinyl, imidazolidinyl, pyrazolidinyl, piperidinyl, piperazinyl, tetrahydrofuranyl, tetrahydropyranyl, morpholinyl, thiomorpholinyl and azepanyl. Examples of bicyclic heterocyclyl groups in which one of the rings is non-aromatic include dihydrobenzofuranyl, indanyl, indolinyl, isoindolinyl, tetrahydroisoquinolinyl, tetrahydroquinolyl, and benzoazepanyl. Examples of monocyclic aromatic heterocyclyl groups (also referred to as monocyclic heteroaryl groups) include furanyl, thienyl, pyrrolyl, oxazolyl, thiazolyl, imidazolyl, oxadiazolyl, thiadiazolyl, pyridyl, triazolyl, triazinyl, pyridazyl, isothiazolyl, isoxazolyl, pyrazinyl, pyrazolyl, and pyrimidinyl. Examples of bicyclic aromatic heterocyclyl groups (also referred to as bicyclic heteroaryl groups) include quinoxalinyl, quinazolinyl, pyridopyrazinyl, benzoxazolyl, benzothiophenyl, benzimidazolyl, naphthyridinyl, quinolinyl, benzofuranyl, indolyl, benzothiazolyl, oxazolyl[4,5-b]pyridyl, pyridopyrimidinyl, isoquinolinyl, and benzohydroxazole.

As used herein, the term “saturated” refers to a group where all available valence bonds of the backbone atoms are attached to other atoms Representative examples of saturated groups include, but are not limited to, butyl, cyclohexyl, piperidine, and the like.

As used herein, the term “substituted” refers to a group having one or more hydrogens or other atoms removed from a carbon or suitable heteroatom and replaced with a further group (i.e., substituent).

As used herein, the term “unsubstituted” refers to a group that does not have any further groups attached thereto or substituted therefore.

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety.

### Prader-Willi Syndrome

The most common endocrinopathy is growth hormone deficiency (GHD), which is reported to occur in 40-100% of patients. Dysregulation of the GH-IGF-1 axis is considered to be universal in PWS (Heksch et al. 2017) and essentially 100% of PWS have subnormal serum IGF-1 levels. The precise etiology of GHD and perturbation of the GH-IGF-1 axis is not yet fully understood. The degree of GH-IGF-1 dysregulation varies to a certain extent with the underlying genetic cause but shows significant variability within group. Recombinant human growth hormone (rhGH) is approved to treat GHD and is often prescribed soon after diagnosis irrespective of whether GH levels are subnormal. Among PWS patients treated with rhGH, IGF-1 levels in peripheral circulation are typically much higher than in children without PWS being treated for primary GHD. Bakker et al. (2015) found that serum IGF Binding Protein-3 (IGFBP3) and acid labile subunit (ALS) were also elevated, suggesting that a significant amount of IGF-1 is sequestered in a IGF-1/ALS/IGFBP3 complex. The extent to which PWS affects endogenous IGF-1 expression and/or bioavailability in the brain is uncertain. Chronic neuroinflammation has also been reported in PWS (Krefft et al. 2020). Significantly elevated levels of interleukin (IL)-1β and IL-13 and significant positive correlations between the levels of IL-1β and a standardised measure of psychopathological behaviours were also found. In a transcriptomic analysis of post-mortem tissues, microglial genes associated with inflammation were upregulated while genes associated with synaptic plasticity and neurogenesis were down-regulated (Bochukova et al. 2018).

PWS is associated with a constellation of symptoms that significantly negatively impact upon quality of life for affected individuals and their families. The initial clinical course of PWS is characterised by hypotonia in infants, with decreased movement, lethargy, feeding difficulties, and failure to thrive. A defining feature of PWS is the change in appetite over time, with the onset of hyperphagia (an unrelenting, pathologically excessive appetite) sometime after early childhood. Whereas infants with PWS do not show normal signs of hunger and often require feeding via nasogastric tubes or other assistive means, feeding improves in young children and subsequently progresses to hyperphagia during childhood. Adolescents and adults with PWS will become morbidly obese if strict environmental controls restricting food intake are not implemented. Additional abnormalities associated with PWS include growth hormone deficiency, hypogonadotropic hypogonadism, sleep disorders, reduced pain sensitivity, poor bone health, decreased gastrointestinal motility, and scoliosis. Aspects such as central adrenal insufficiency, seizures, hypothyroidism, and hypoglycaemia occur at frequencies higher than the unaffected population, but are not present in all individuals (Bohonowych et al. 2019).

In addition to somatic symptoms, intellectual disability (ID) and neuropsychiatric issues are present to some degree in all individuals with PWS. Individuals with PWS typically exhibit a characteristic behavioural phenotype that includes cognitive rigidity, heightened anxiety, severe temper outbursts, obsessive-compulsive behaviours, and self-injurious behaviours. Adolescents and adults are at risk of mental illness and autistic symptomatology is common, particularly in those with PWS by UPD. Hyperphagia-driven behaviours include food-seeking behaviour, hoarding or foraging for food, eating of inappropriate food items, stealing food or money to buy food, and intense psychological stress and behavioural disturbances associated with food denial. Behavioural issues and an inability to control food intake represent the major impediments to independent living for individuals with PWS, and opportunities for community engagement, employment, independent living, and social activities are highly constrained by these issues (Bohonowych et al. 2019).

### Methods of Treatment

The present disclosure is directed to providing compounds useful in the treatment of Prader-Willi Syndrome.

In one aspect, there is provided a method of treating Prader-Willi Syndrome in a subject, comprising administering to the subject a therapeutically effective amount of a compound of Formula I, or a pharmaceutically acceptable salt, hydrate, or prodrug thereof:

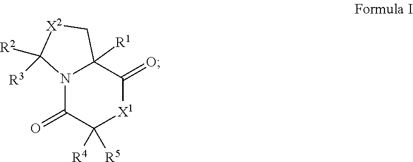

wherein:
X¹ is selected from the group consisting of NR′, O, and S;
X² is selected from the group consisting of CH₂, NR′, O, and S;
R¹, R², R³, R⁴, and R⁵ are independently selected from the group consisting of —H, halogen, —OR′, —SR′, —NR′R′, —NO₂, —CN, —C(O)R′, —C(O)OR′, —C(O)NR′R′, —C(NR′)NR′R′, -alkyl, heteroalkyl, alkenyl, alkynyl, 3-10-membered carbocycle, and 3-10-membered heterocycle,
   wherein each alkyl, heteroalkyl, alkenyl, alkynyl, 3-10-membered carbocycle, and 3-10-membered heterocycle is unsubstituted or substituted with one or more substituents selected from the group consisting of halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, and alkynyl;
   wherein each R′ is independently selected from the group consisting of hydrogen, alkyl, heteroalkyl, alkenyl, alkynyl, 3-10-membered carbocycle, and 3-10-membered heterocycle;
or R⁴ and R⁵ taken together are —CH₂—(CH₂)ₙ—CH₂— where n is an integer from 0-6;
or R² and R³ taken together are —CH₂—(CH₂)ₙ—CH₂— where n is an integer from 0-6.

In some embodiments of the above aspect, there is provided a proviso that when R¹ is CH₃, R² is hydrogen, R³ is hydrogen, and R⁴ is hydrogen, then R⁵ is not benzyl; and when R¹ is hydrogen, then at least one of R² and R³ is not hydrogen.

In one embodiment, the method of treatment provides the treatment of one or more symptoms associated with an abnormality in chromosome 15. In one example, the method of treatment provides the treatment of one or more symptoms associated with Prader-Willi Syndrome.

Numerous symptoms are associated with Prader-Willi Syndrome. Examples of somatic symptoms include, but are not limited to, early failure to thrive, excessive appetite (hyperphagia), obesity, type 2 diabetes, metabolic syndrome, multiple endocrine abnormalities, hypotonia, hypogonadism, sleep disturbances, sleep apnoea, speech disorders, reduced pain sensitivity, poor bone health, strabismus, depigmentation, decreased gastrointestinal motility, scoliosis, adrenal insufficiencies, seizures, hypothyroidism, hypoglycaemia, hypogonadotropic hypogonadism, and distinctive facial features.

Accordingly, in some embodiments, there is provided a method of treating a symptom or finding associated with Prader-Willi Syndrome in a subject, comprising administering to the subject a therapeutically effective amount of a compound of Formula I, or a pharmaceutically acceptable salt, hydrate, or prodrug thereof, as described herein, wherein the symptom is selected from the group consisting of early failure to thrive, excessive appetite (hyperphagia), obesity, type 2 diabetes, metabolic syndrome, multiple endocrine abnormalities, hypotonia, hypogonadism, sleep disturbances, sleep apnoea, speech disorders, reduced pain sensitivity, poor bone health, strabismus, depigmentation, decreased gastrointestinal motility, scoliosis, adrenal insufficiencies, seizures, hypothyroidism, hypoglycaemia, hypogonadotropic hypogonadism, and distinctive facial features. In one example, the symptom associated with Prader-Willi Syndrome is hyperphagia. In one example, the symptom associated with Prader-Willi Syndrome is obesity. In one example, the finding associated with Prader-Willi Syndrome is elevated blood insulin. In one example, the symptom associated with Prader-Willi Syndrome is hyperphagia.

In addition to somatic symptoms, intellectual disability and neuropsychiatric issues may present as symptoms of Prader-Willi Syndrome. In some embodiments, the intellectual disability and/or neuropsychiatric symptoms are selected from the group consisting of mild to moderate intellectual and learning disabilities, cognitive impairment, neurobehavioural disorders, intellectual disability, cognitive rigidity, heightened anxiety, severe temper outbursts, obsessive-compulsive behaviours, and self-injurious behaviours. Adolescents and adults are at risk of mental illness and autistic symptomatology.

Accordingly, in some embodiments, there is provided a method of treating a symptom associated with Prader-Willi Syndrome in a subject, comprising administering to the subject a therapeutically effective amount of a compound of Formula I, or a pharmaceutically acceptable salt, hydrate, stereoisomer, or prodrug thereof, as described herein, wherein the symptom is selected from the group consisting of mild to moderate intellectual and learning disabilities, cognitive impairment, neurobehavioural disorders, intellectual disability, cognitive rigidity, heightened anxiety, severe temper outbursts, obsessive-compulsive behaviours, self-injurious behaviours, mental illness, and autism.

The most common endocrinopathy is growth hormone deficiency (GHD), which results in IGF-1 deficiency. In one example, the symptom associated with Prader-Willi Syndrome is IGF-1 deficiency. In some embodiments, there is provided a method of treating a symptom associated with Prader-Willi Syndrome in a subject, comprising administering to the subject a therapeutically effective amount of a compound of Formula I, or a pharmaceutically acceptable salt, hydrate, or prodrug thereof, as described herein, wherein the symptom is IGF-1 deficiency.

In a further aspect, there is provided the use of a compound of Formula I, or a pharmaceutically acceptable salt, hydrate, stereoisomer, or prodrug thereof:

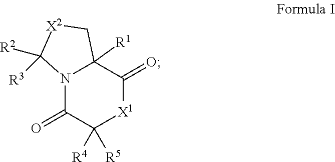

wherein:
X¹ is selected from the group consisting of NR′, O, and S;
X² is selected from the group consisting of CH₂, NR′, O, and S;
R¹, R², R³, R⁴, and R⁵ are independently selected from the group consisting of —H, halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, alkynyl, 3-10-membered carbocycle, and 3-10-membered heterocycle,
   wherein each alkyl, heteroalkyl, alkenyl, alkynyl, 3-10-membered carbocycle, and 3-10-membered heterocycle is unsubstituted or substituted with one or more substituents selected from the group consisting of halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, and alkynyl;
   wherein each R′ is independently selected from the group consisting of hydrogen, alkyl, heteroalkyl, alkenyl, alkynyl, 3-10-membered carbocycle, and 3-10-membered heterocycle;
or R⁴ and R⁵ taken together are —CH₂—(CH₂)ₙ—CH₂— where n is an integer from 0-6;
or R² and R³ taken together are —CH₂—(CH₂)ₙ—CH₂— where n is an integer from 0-6;
in the manufacture of a medicament for the treatment of Prader-Willi Syndrome.

In some embodiments of the above aspect, there is provided a proviso that when R¹ is CH₃, R² is hydrogen, R³ is hydrogen, and R⁴ is hydrogen, then R⁵ is not benzyl; and when R¹ is hydrogen, then at least one of R² and R³ is not hydrogen.

In a further aspect, there is provided a compound of Formula I, or a pharmaceutically acceptable salt, hydrate, stereoisomer, or prodrug thereof:

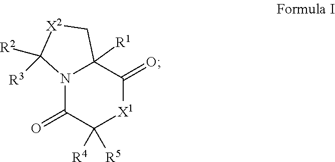

wherein:
X¹ is selected from the group consisting of NR′, O, and S;
X² is selected from the group consisting of CH₂, NR′, O, and S;
R¹, R², R³, R⁴, and R⁵ are independently selected from the group consisting of —H, halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, alkynyl, 3-10-membered carbocycle, and 3-10-membered heterocycle,
   wherein each alkyl, heteroalkyl, alkenyl, alkynyl, 3-10-membered carbocycle, and 3-10-membered heterocycle is unsubstituted or substituted with one or more substituents selected from the group consisting of halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, and alkynyl;
   wherein each R′ is independently selected from the group consisting of hydrogen, -alkyl, heteroalkyl, alkenyl, alkynyl, 3-10-membered carbocycle, and 3-10-membered heterocycle;
or R⁴ and R⁵ taken together are —CH₂—(CH₂)ₙ—CH₂— where n is an integer from 0-6;
or R² and R³ taken together are —CH₂—(CH₂)ₙ—CH₂— where n is an integer from 0-6;
for use in treating Prader-Willi Syndrome.

In some embodiments of the above aspect, there is provided a proviso that when R¹ is CH₃, R² is hydrogen, R³ is hydrogen, and R⁴ is hydrogen, then R⁵ is not benzyl; and when R¹ is hydrogen, then at least one of R² and R³ is not hydrogen.

One aspect is a method or use of any preceding or following aspect for treating Prader-Willi Syndrome in a subject, comprising administering to the subject a therapeutically effective amount of a compound of Formulas I, II, III or IV, or a pharmaceutically acceptable salt, hydrate, stereoisomer, or prodrug thereof:

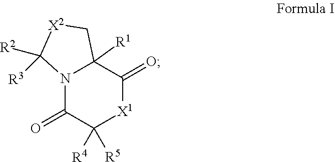

wherein:
X¹ is selected from the group consisting of NR′, O, and S;
X² is selected from the group consisting of CH₂, NR′, O, and S;
R¹, R², R³, R⁴, and R⁵ are independently selected from the group consisting of hydrogen, halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, alkynyl, 3-10-membered carbocycle, and 3-10-membered heterocycle,
   wherein each alkyl, heteroalkyl, alkenyl, alkynyl, 3-10-membered carbocycle, and 3-10-membered heterocycle is unsubstituted or substituted with one or more substituents selected from the group consisting of halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, and alkynyl;
   wherein each R′ is independently selected from the group consisting of hydrogen, alkyl, heteroalkyl, alkenyl, alkynyl, 3-10-membered carbocycle, and 3-10-membered heterocycle;
or R⁴ and R⁵ taken together are —CH₂—(CH₂)ₙ—CH₂— where n is an integer from 0-6;
or R² and R³ taken together are —CH₂—(CH₂)ₙ—CH₂— where n is an integer from 0-6.

In some embodiments of the above aspect, there is provided a proviso that when R¹ is CH₃, R² is hydrogen, R³ is hydrogen, and R⁴ is hydrogen, then R⁵ is not benzyl; and when R¹ is hydrogen, then at least one of R² and R³ is not hydrogen.

One aspect is a method or use of any preceding or following aspect, wherein R¹ is selected from the group consisting of hydrogen, —CH₃, and —CH₂CHCH₂.

One aspect is a method or use of any preceding or following aspect, wherein R² is selected from the group consisting of hydrogen and —CH₃.

One aspect is a method or use of any preceding or following aspect, wherein R³ is selected from the group consisting of hydrogen and —CH₃.

One aspect is a method or use of any preceding or following aspect, wherein X¹ is NH.

One aspect is a method or use of any preceding or following aspect, wherein X² is selected from the group consisting of CH₂ and S.

One aspect is a method or use of any preceding or following aspect, wherein R⁴ and R⁵ are each hydrogen, or taken together are selected from the group consisting of —CH₂—(CH₂)₃—CH₂— and —CH₂—(CH₂)₂—CH₂—.

It will be appreciated that any one or more of the above aspects, embodiments and/or examples may be combined with each other to provide any one or more further aspects, embodiments and/or examples. For example, in the above Formula I: R¹ is alkyl or alkenyl; R² is hydrogen; R³ is hydrogen; R⁴ and R⁵ are each hydrogen or taken together are C₃₋₁₀carbocycle; X¹ is NH; X² is CH₂; wherein each alkyl, alkenyl, and C₃₋₁₀carbocycle, is unsubstituted or substituted with one or more substituents selected from the group consisting of halogen, —OR′, —SR′, —NR′R′, —NO₂, —CN, —C(O)R′, —C(O)OR′, —C(O)NR′R′, —C(NR′)NR′R′, alkyl, haloalkyl, alkenyl, and haloalkenyl; and wherein each R′ is independently selected from the group consisting of hydrogen, alkyl, and alkenyl. In a further example, R¹ is C₁₋₆alkyl (e.g. —CH₃) or C₂₋₆alkenyl (e.g. —CH₂—CH═CH₂). In a further example, R⁴ and R⁵ taken together are a monocyclic saturated C₃₋₁₀carbocycle, which can be substituted or unsubstituted as defined above or herein. The unsubstituted or substituted monocyclic saturated C₃₋₁₀carbocycle may be an unsubstituted or substituted cyclopentyl or cyclohexyl group. In another example, the C₃₋₁₀carbocycle is substituted with one or more substituents selected from the group consisting of halogen, OH, —NO₂, —NH₂, —CN, —C(O)H, —C(O)OH, —C(O)NH₂, alkyl, haloalkyl, alkenyl, and haloalkenyl.

One aspect is a method or use of any preceding or following aspect, wherein the compound of Formula I is selected from the group consisting of:

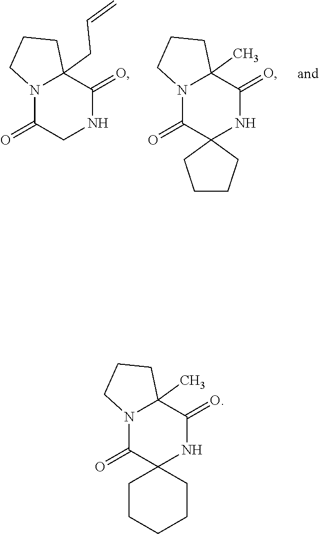

One aspect is a method or use of any preceding or following embodiments, wherein the compound of Formula I is:

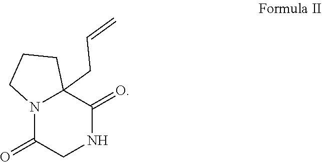

One aspect is a method or use of any preceding or following aspect, wherein the treatment comprises preventing or reducing the likelihood or severity of one or more symptoms of Prader-Willi Syndrome.

One aspect is a method or use of any preceding or following aspect, wherein Prader-Willi Syndrome is assessed using one or more clinical tests selected from the group consisting of genetic testing, IGF-1 in serum, Total IGF-1, Free IGF-1, Bound (to IGFBPs) IGF-1, IGF-1 in cerebral spinal fluid (CSF), Total IGF-1, Free IGF-1, IGFBPs in serum, IGFBP-1, -2, -3, -4, -5, -6 in serum, IGFBPs in CSF, IGFBP-1, -2, -3, -4, -5, -6 in CSF, blood glucose, blood lipids (HDL, LDL, VLDL, triglycerides), Homeostatic Model Assessment for Insulin Resistance (HOMA-IR), Body mass index (BMI), and Body fat assessment (BFA).

One aspect is a method or use of any preceding or following aspect, wherein the severity of the symptom is assessed using one or more clinical tests selected from the group consisting of the Hyperphagia Questionnaire for Clinical Trials (HQ-CT), the Clinical Global Impression of Severity (CGI-S), the Clinical Global Impression of Change (CGI-I), the Caregiver Global Impression of Change (CaGI-I), the Aberrant Behavior Checklist (ABC) and ABC Subscales, the Social Responsiveness Scale, the Repetitive Behavior Scale-Revised (RBS-R), the PWS Anxiety and Distress Questionnaire (PADQ), Intestinal microbiota composition (16S or other sequencing method), the Children's Yale-Brown Obsessive-Compulsive Scale (CY-BOCS), and the Montefiore Einstein Rigidity Scale-Revised-PWS (MERS-R-PWS).

One aspect is a method or use of any preceding or following aspect, wherein the symptom associated with Prader-Willi Syndrome is selected from the group consisting of early failure to thrive, excessive appetite (hyperphagia), obesity, type 2 diabetes, metabolic syndrome, multiple endocrine abnormalities, hypotonia, hypogonadism, sleep disturbances, sleep apnoea, speech disorders, reduced pain sensitivity, poor bone health, strabismus, depigmentation, decreased gastrointestinal motility, scoliosis, adrenal insufficiencies, seizures, hypothyroidism, hypoglycaemia, hypogonadotropic hypogonadism, distinctive facial features, mild to moderate intellectual and learning disabilities, cognitive impairment, neurobehavioural disorders, intellectual disability, cognitive rigidity, heightened anxiety, severe temper outbursts, obsessive-compulsive behaviours, self-injurious behaviours, mental illness, autistic symptomatology, and growth hormone deficiency (GHD).

One aspect is a method or use of any preceding or following aspect, wherein the compound of Formulas I, II, III or IV is administered in combination with a therapeutic agent.

One aspect is a method or use of any preceding or following aspect, wherein the therapeutic agent is selected from the group consisting of recombinant human growth hormone (rhGH), human growth hormone, recombinant human IGF-1 (rhIGF-1), IGF-1, IGF-2, any IGF Binding Protein (IGFBP), IGFBP-3, insulin, any statin, any appetite suppressant, transforming growth factor-β1, activin, nerve growth factor, growth hormone binding protein, basic fibroblast growth factor, acidic fibroblast growth factor, the hst/Kfgk gene product, FGF-3, FGF-4, FGF-6, keratinocyte growth factor, androgen-induced growth factor, int-2, fibroblast growth factor, homologous factor-1 (FHF-1), FHF-2, FHF-3, FHF-4, keratinocyte growth factor 2, glial activating factor, FGF-10, FGF-16, ciliary neurotrophic factor, brain derived nerve growth factor, neurotrophin 3, neurotrophin 4, bone morphogenetic protein 2 (BMP-2), glial-cell line derived neurotrophic factor, activity-dependant neurotrophic factor, cytokine leukaemia inhibiting factor, oncostatin M, an interleukin, a-interferon, β-interferon, γ-interferon, consensus interferon, TNF-a, clomethiazole; kynurenic acid, Semax, tacrolimus, L-threo-1-phenyl-2-decanoylamino3, 3-morpholino-1-propanol, adrenocorticotropin-(4-9) analogue (ORG 2766), dizolcipine [MK-801], selegiline, NPS1506, GV1505260, MK-801, GV150526, 2,3-dihydroxy-6-nitro-7-sulfamoylbenzo(f)quinoxaline (NBQX), LY303070, LY300164, and the anti-MAdCAM-1 antibody MECA-367.

One aspect is a method or use of any preceding or following aspect, wherein the therapeutic agent is recombinant human growth hormone (rhGH).

One aspect is a method or use of any preceding or following aspect, wherein the pharmaceutical composition is administered orally.

One aspect is a method or use of any preceding or following aspect, wherein the compound of Formulas I, II, III or IV is administered in a dosage of from about 0.001 mg/kg to and including about 600 mg/kg.

One aspect is a method or use of any preceding or following aspect, wherein the subject is a mammal.

One aspect is a method or use of any preceding or following aspect, wherein the subject is a human.

One aspect is a method or use of any preceding or following aspect, wherein the compound of Formulas I, II, III or IV is administered in the form of a pharmaceutical composition comprising a pharmaceutically acceptable excipient.

One aspect is a method or use of any preceding or following aspect, wherein the pharmaceutically acceptable excipient is selected from the group consisting of binders, carriers, additives, adjuvants, microemulsions, coarse emulsions, and liquid crystals.

One aspect is a method or use of any preceding or following aspect, wherein the pharmaceutical composition is formulated as an oral solution, an oral suspension, or as a powder for preparing an oral solution or oral suspension.

One aspect is a method or use of any preceding or following aspect, wherein the pharmaceutical composition is formulated as a tablet or a capsule

One aspect is a compound of one of Formulas I, II, III or IV, or a pharmaceutically acceptable salt, hydrate, stereoisomer, or prodrug thereof:

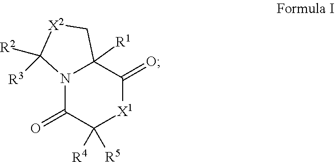

wherein:
X¹ is selected from the group consisting of NR′, O, and S;
X² is selected from the group consisting of CH₂, NR′, O, and S;
R¹, R², R³, R⁴, and R⁵ are independently selected from the group consisting of hydrogen, halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, alkynyl, 3-10-membered carbocycle, and 3-10-membered heterocycle,
   wherein each alkyl, heteroalkyl, alkenyl, alkynyl, 3-10-membered carbocycle, and 3-10-membered heterocycle is unsubstituted or substituted with one or more substituents selected from the group consisting of halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, and alkynyl;
   wherein each R′ is independently selected from the group consisting of hydrogen, alkyl, heteroalkyl, alkenyl, alkynyl, 3-10-membered carbocycle, and 3-10-membered heterocycle;
or R⁴ and R⁵ taken together are —CH₂—(CH₂)ₙ—CH₂— where n is an integer from 0-6;
or R² and R³ taken together are —CH₂—(CH₂)ₙ—CH₂— where n is an integer from 0-6;
with the proviso that when R¹ is CH₃, R² is hydrogen, R³ is hydrogen, and R⁴ is hydrogen, then R⁵ is not benzyl; and when R¹ is hydrogen, then at least one of R² and R³ is not hydrogen;
   in the manufacture of a medicament for the treatment of Prader-Willi Syndrome.

One aspect is a compound of one of Formulas I, II, III or IV, or a pharmaceutically acceptable salt, hydrate, stereoisomer, or prodrug thereof:

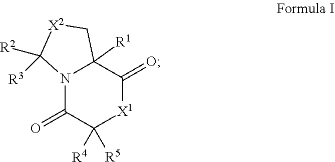

wherein:
X¹ is selected from the group consisting of NR′, O, and S;
X² is selected from the group consisting of CH₂, NR′, O, and S;
R¹, R², R³, R⁴, and R⁵ are independently selected from the group consisting of hydrogen, halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, alkynyl, 3-10-membered carbocycle, and 3-10-membered heterocycle,
   wherein each alkyl, heteroalkyl, alkenyl, alkynyl, 3-10-membered carbocycle, and 3-10-membered heterocycle is unsubstituted or substituted with one or more substituents selected from the group consisting of halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, and alkynyl;
   wherein each R′ is independently selected from the group consisting of hydrogen, alkyl, heteroalkyl, alkenyl, alkynyl, 3-10-membered carbocycle, and 3-10-membered heterocycle;
or R⁴ and R⁵ taken together are —CH₂—(CH₂)ₙ—CH₂— where n is an integer from 0-6;
or R² and R³ taken together are —CH₂—(CH₂)ₙ—CH₂— where n is an integer from 0-6;
with the proviso that when R¹ is CH₃, R² is hydrogen, R³ is hydrogen, and R⁴ is hydrogen, then R⁵ is not benzyl; and when R¹ is hydrogen, then at least one of R² and R³ is not hydrogen;
   for use in treating Prader-Willi Syndrome.

One aspect is a use of a compound of any of Formulas I, II, III or IV for treating a subject having Prader-Willi Syndrome as described herein.

One aspect is a method of any preceding or following aspect, wherein said subject is a human being.

One aspect is a use of any preceding aspect, wherein said subject is a human being.

One aspect is a method or use of any preceding or following aspect, wherein said compound is cG-2-AllylP, or Cyclic Cyclopentyl-G-2-MeP, or Cyclic Cyclohexyl-G-2-MeP.

One aspect is a method or use of any preceding or following aspect,

wherein said compound is formulated in an aqueous solution.

One aspect is a method or use of any preceding or following aspect, wherein said compound is cG-2-AllylP, or Cyclic Cyclopentyl-G-2-MeP, or Cyclic Cyclohexyl-G-2-MeP.

One aspect is a method or use of any preceding aspect, wherein said compound is formulated in an aqueous solution.

Other aspects include the method of any preceding or following aspect, wherein the dose of said compound is from about 0.01 mg per kg of body weight (mg/kg) to about 1000 mg/kg, alternatively from about 0.1 mg/kg to about 500 mg/kg., or about 0.1 mg/kg. to about 200 mg/kg, or about 0.01, or 0.1, or 1, or 10, or 20, or 50, or 75, or 100, or 500, or 1000, or 5000 mg/kg, respectively.

### Clinical Assessment

Prader-Willi Syndrome may be assessed using clinical tests including, for example, genetic testing, IGF-1 in serum, Total IGF-1, Free IGF-1, Bound (to IGFBPs) IGF-1, IGF-1 in cerebral spinal fluid (CSF), Total IGF-1, Free IGF-1, IGFBPs in serum, IGFBP-1, -2, -3, -4, -5, -6 in serum, IGFBPs in CSF, IGFBP-1, -2, -3, -4, -5, -6 in CSF, blood glucose, blood lipids (HDL, LDL, VLDL, triglycerides), Homeostatic Model Assessment for Insulin Resistance (HOMA-IR), Body mass index (BMI), and Body fat assessment (BFA).

The severity of the symptoms associated with Prader-Willi Syndrome may also be assessed using one or more clinical tests including, for example, the Hyperphagia Questionnaire for Clinical Trials (HQ-CT), the Clinical Global Impression of Severity (CGI-5), the Clinical Global Impression of Change (CGI-I), the Caregiver Global Impression of Change (CaGI-I), the Aberrant Behavior Checklist (ABC) and ABC Subscales, the Social Responsiveness Scale, the Repetitive Behavior Scale-Revised (RBS-R), the PWS Anxiety and Distress Questionnaire (PADQ), Intestinal microbiota composition (16S or other sequencing method), the Children's Yale-Brown Obsessive-Compulsive Scale (CY-BOCS), and the Montefiore Einstein Rigidity Scale-Revised-PWS (MERS-R-PWS).

### Compounds of Formula I

As described herein, the use or method of treating Prader-Willi Syndrome comprises a compound of Formula I, or a pharmaceutically acceptable salt, hydrate, stereoisomer, or prodrug thereof:

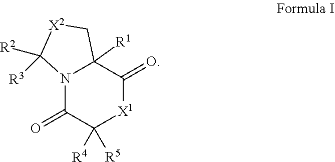

In the above Formula I, X¹ is selected from the group consisting of NR′, O, and S. In one embodiment, X¹ is NR′. In one example, X¹ is NH. In one example, X¹ is O. In one example, X¹ is S.

In the above Formula I, X² is selected from the group consisting of CH₂, NR′, O, and S. In one example, X² is CH₂. In one embodiment, X² is NR′. In one example, X² is NH. In one example, X² is O. In one example, X² is S.

In the above Formula I, R¹, R², R³, R⁴, and R⁵ are independently selected from the group consisting of hydrogen, halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, alkynyl, 3-10-membered carbocycle, and 3-10-membered heterocycle.

That is, in the above Formula I, R¹ is selected from the group consisting of hydrogen, halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, alkynyl, 3-10-membered carbocycle, 3-10-membered heterocycle. In one embodiment, R¹ is alkyl. In one embodiment, R¹ is C₁₋₆alkyl. In one example, R¹ is CH₃. In one embodiment, R¹ is alkenyl. In one embodiment, R¹ is C₂₋₆alkenyl. In one example, R¹ is —CH₂CHCH₂.

In the above Formula I, R² is selected from the group consisting of hydrogen, halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, alkynyl, 3-10-membered carbocycle, and 3-10-membered heterocycle. In one example, R² is hydrogen. In one embodiment, R² is alkyl. In one embodiment, R² is C₁₋₆-alkyl. In one example, R² is CH₃.

In the above Formula I, R³ is selected from the group consisting of hydrogen, halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, alkynyl, 3-10-membered carbocycle, and 3-10-membered heterocycle. In one example, R³ is hydrogen. In one embodiment, R³ is alkyl. In one embodiment, R³ is C₁₋₆-alkyl. In one example, R³ is CH₃.

In the above Formula I, R⁴ is selected from the group consisting of hydrogen, halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, alkynyl, 3-10-membered carbocycle, and 3-10-membered heterocycle. In one example, R⁴ is hydrogen.

In the above Formula I, R⁵ is selected from the group consisting of hydrogen, halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, alkynyl, 3-10-membered carbocycle, and 3-10-membered heterocycle. In one example, R⁵ is hydrogen.

In the above Formula I, in one embodiment, R⁴ and R⁵ taken together are —CH₂—(CH₂)ₙ—CH₂—, where n is 0, 1, 2, 3, 4, 5, or 6. In one example, R⁴ and R⁵ taken together are —CH₂—(CH₂)₂—CH₂—. That is, in one example, n is 2. In one example, R⁴ and R⁵ taken together are —CH₂—(CH₂)₃—CH₂—. That is, in one example, n is 3. In one example, R⁴ and R⁵ are both hydrogen.

In the above Formula I, each R′ is independently selected from the group consisting of hydrogen, alkyl, heteroalkyl, alkenyl, alkynyl, 3-10-membered carbocycle, and 3-10-membered heterocycle. In one example, R′ is hydrogen, alkyl, or alkenyl. In one example, R′ is hydrogen.

In the above Formula I, when R¹ is CH₃ and R² is hydrogen, and R³ is hydrogen and R⁴ is hydrogen, then R⁵ is not benzyl. In the above Formula I, when R¹ is hydrogen, then at least one of R² and R³ is not hydrogen.

It will be appreciated that any one or more of the above aspects, embodiments and examples may be combined with each other to provide any one or more further specific embodiments and examples. For example, in the above Formula I: R¹ is alkyl or alkenyl; R² is hydrogen; R³ is hydrogen; R⁴ and R⁵ are each hydrogen or taken together are C₃₋₁₀carbocycle; X¹ is NH; X² is CH₂; wherein each alkyl, alkenyl, and C₃₋₁₀carbocycle, is unsubstituted or substituted with one or more substituents selected from the group consisting of halogen, —OR′, —SR′, —NR′R′, —NO₂, —CN, —C(O)R′, —C(O)OR′, —C(O)NR′R′, —C(NR′)NR′R′, alkyl, haloalkyl, alkenyl, and haloalkenyl; and wherein each R′ is independently selected from the group consisting of hydrogen, alkyl, and alkenyl. In a further example, R¹ is C₁₋₆alkyl (e.g. —CH₃) or C₂₋₆alkenyl (e.g. —CH₂—CH═CH₂). In a further example, R⁴ and R⁵ taken together are a monocyclic saturated C₃₋₁₀carbocycle, which can be substituted or unsubstituted as defined above or herein. The unsubstituted or substituted monocyclic saturated C₃₋₁₀carbocycle may be an unsubstituted or substituted cyclopentyl or cyclohexyl group. In another example, the C₃₋₁₀carbocycle is substituted with one or more substituents selected from the group consisting of halogen, OH, —NO₂, —NH₂, —CN, —C(O)H, —C(O)OH, —C(O)NH₂, alkyl, haloalkyl, alkenyl, and haloalkenyl.

In one example, the compound of Formula I is selected from the group consisting of:

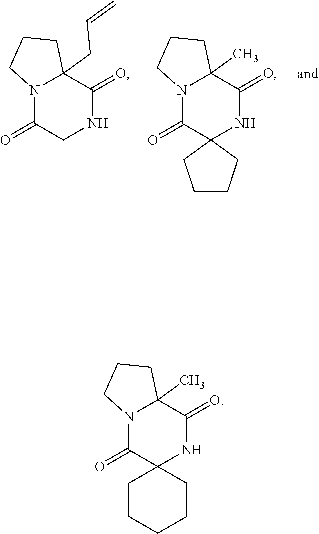

In one example, the compound of Formula I is:

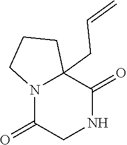

### “Cyclic Glycyl-2-Allyl Proline” or “Cyclic G-2-AllylP” or “NNZ2591” Formula II

That is, in one example, in the compound of Formula I, X¹ is NH; X² is CH₂; R¹ is —CH₂CHCH₂; and R², R³, R⁴, and R⁵ are each hydrogen.

In one example, the compound of Formula I is:

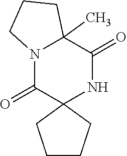 [SMILES: CC12CCCN1C(=O)C1(CCCC1)NC2=O]

### “Cyclic cyclopentyl-G-2-MeP” Formula III

That is, in one example, in the compound of Formula I, X¹ is NH; X² is CH₂; R¹ is CH₃; R² and R³ are each hydrogen; and R⁴ and R⁵ taken together are —CH₂—(CH₂)₂—CH₂—.

In one example, the compound of Formula I is:

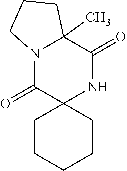 [SMILES: CC12CCCN1C(=O)C1(CCCCC1)NC2=O]

### “Cyclic cyclohexyl-G-2-MeP” Formula IV

That is, in one example, in the compound of Formula I, X¹ is NH; X² is CH₂; R¹ is CH₃; R² and R³ are each hydrogen; and R⁴ and R⁵ taken together are —CH₂—(CH₂)₃—CH₂—.

### Pharmaceutical Compositions

In some embodiments, there is provided the use or method as described herein, wherein the compound of Formulas I, II, III, or IV, or pharmaceutically acceptable salts, hydrates, stereoisomers, or prodrugs thereof, is administered in the form of a pharmaceutical composition.

Suitably, the pharmaceutically acceptable carrier, diluent and/or excipient may be or include one or more of diluents, solvents, pH buffers, binders, carriers, additives, adjuvants, microemulsions, coarse emulsions, liquid crystals, fillers, emulsifiers, disintegrants, polymers, lubricants, oils, fats, waxes, coatings, viscosity-modifying agents, glidants and the like. In some embodiments, there is provided the method as described herein, wherein the compound of Formula I, or pharmaceutically acceptable salt, hydrate, or prodrug thereof, is administered in the form of a pharmaceutical composition comprising a pharmaceutically acceptable excipient.

Diluents may include one or more of microcrystalline cellulose, lactose, mannitol, calcium phosphate, calcium sulfate, kaolin, dry starch, powdered sugar, and the like. Binders may include one or more of povidone, starch, stearic acid, gums, hydroxypropylmethyl cellulose and the like. Disintegrants may include one or more of starch, croscarmellose sodium, crospovidone, sodium starch glycolate and the like. Solvents may include one or more of ethanol, methanol, isopropanol, chloroform, acetone, methylethyl ketone, methylene chloride, water and the like. Lubricants may include one or more of magnesium stearate, zinc stearate, calcium stearate, stearic acid, sodium stearyl fumarate, hydrogenated vegetable oil, glyceryl behenate and the like. A glidant may be one or more of colloidal silicon dioxide, talc or cornstarch and the like. Buffers may include phosphate buffers, borate buffers and carbonate buffers, although without limitation thereto. Fillers may include one or more gels inclusive of gelatin, starch and synthetic polymer gels, although without limitation thereto. Coatings may comprise one or more of film formers, solvents, plasticizers and the like. Suitable film formers may be one or more of hydroxypropyl methyl cellulose, methyl hydroxyethyl cellulose, ethyl cellulose, hydroxypropyl cellulose, povidone, sodium carboxymethyl cellulose, polyethylene glycol, acrylates and the like. Suitable solvents may be one or more of water, ethanol, methanol, isopropanol, chloroform, acetone, methylethyl ketone, methylene chloride and the like. Plasticizers may be one or more of propylene glycol, castor oil, glycerin, polyethylene glycol, polysorbates, and the like.

Reference is made to the Handbook of Pharmaceutical Excipients 9th Edition, Eds. Sheskey, Hancock, Moss & Goldfarb (2020), which provides non-limiting examples of excipients which may be useful according to the present disclosure. Other pharmaceutical excipients and/or additives suitable for use in the compositions according to the present disclosure are listed in “Remington: The Science & Practice of Pharmacy”, 23^{rd} Edition, Ed. Adejare (2020), and in the “Prescribers' Digital Reference”, at www._pdr._net (2021, and in “Handbook of Pharmaceutical Excipients”, Third Ed., Ed. A. H. Kibbe, Pharmaceutical Press, 2000.

It will be appreciated that the choice of pharmaceutically acceptable carriers, diluents and/or excipients will, at least in part, be dependent upon the mode of administration of the formulation. By way of example only, the composition may be in the form of a tablet, capsule, caplet, powder, an oral solution or suspension, a powder for preparing an oral solution or suspension, an injectable liquid, a suppository, a slow-release formulation, an osmotic pump formulation, or any other form that is effective and safe for administration.

Examples of pharmaceutical formulations include those suitable for oral, parenteral (including subcutaneous, intradermal, intramuscular, intravenous, and intraarticular), inhalation (including fine particle dusts or mists that may be generated by means of various types of metered dose pressurised aerosols), nebulisers or insufflators, rectal, intraperitoneal and topical (including dermal, buccal, sublingual, and intraocular) administration, although the most suitable route may depend upon, for example, the condition and disorder of the recipient. In one example, the compound of Formula I, or pharmaceutically acceptable salt, hydrate, or prodrug thereof, is administered orally.

The pharmaceutical formulations may conveniently be presented in unit dosage form and may be prepared by any of the methods well known in the art of pharmacy. All methods include the step of bringing a compound of Formula I, pharmaceutically acceptable salt, hydrate, or prodrug thereof, into association with the excipient that constitutes one or more necessary ingredients. In general, the formulations are prepared by uniformly and intimately bringing into association the active ingredient with liquid carriers or finely divided solid carriers or both, and then, if necessary, shaping the product into the desired formulation.

In some embodiments, that composition is formulated for oral delivery. For example, pharmaceutical formulations of the present disclosure suitable for oral administration may be presented as discrete units such as capsules, cachets, pills or tablets each containing a predetermined amount of the active ingredient; as a powder or granules, as a solution or a suspension in an aqueous liquid or non-aqueous liquid, for example as elixirs, tinctures, suspensions or syrups; or as an oil-in-water liquid emulsion or a water-in-oil liquid emulsion. A compound of Formula I, or pharmaceutically acceptable salt, hydrate, or prodrug thereof, may also be presented as a bolus, electuary or paste. In one example, a compound of Formula I, a pharmaceutically acceptable salt, hydrate, or prodrug thereof, is administered as an oral tablet. In one example, a compound of Formula I, a pharmaceutically acceptable salt, hydrate, or prodrug thereof, is administered as an oral capsule. In one example, a compound of Formula I, a pharmaceutically acceptable salt, hydrate, or prodrug thereof, is administered as an oral solution. In one example, a compound of Formula I, a pharmaceutically acceptable salt, hydrate, or prodrug thereof, is administered as an oral suspension.

A tablet may be made, for example, by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active, or dispersing agent. Moulded tablets may be made by moulding in a suitable machine a mixture of the powdered compound moistened with an inert liquid diluent. The tablets may be optionally coated or scored, and may be formulated so as to provide slow or controlled release of the compound of Formula I, or pharmaceutically acceptable salt, hydrate, or prodrug thereof. The compound of Formula I, or pharmaceutically acceptable salt, hydrate, or prodrug thereof, can, for example, be administered in a form suitable for immediate release or extended release. Immediate release or extended release can be achieved by the use of suitable pharmaceutical compositions comprising a compound of Formula I, or pharmaceutically acceptable salt, hydrate, or prodrug thereof, or, particularly in the case of extended release, by the use of devices such as subcutaneous implants or osmotic pumps. A compound of Formula I, or pharmaceutically acceptable salt, hydrate, or prodrug thereof, may also be administered liposomally.

It should be understood that in addition to the ingredients particularly mentioned above, the formulations may include other agents conventional in the art having regard to the type of formulation in question, for example, those suitable for oral administration may include flavouring agents. In one example, a compound of Formulas I, II, III, or IV, or pharmaceutically acceptable salt, hydrate, or prodrug thereof, is formulated as an oral solution. In one example, a compound of Formulas I, II, III, or IV, or a pharmaceutically aceptable salt, hydrate, or prodrug thereof, is formulated as an oral suspension. In one example, a compound of Formulas I, II, III, or IV, or a pharmaceutically aceptable salt, hydrate, or prodrug thereof, is formulated as a powder for preparing an oral solution or oral suspension.

### Solvates and Hydrates

It may be convenient or desirable to prepare, purify and/or handle a corresponding solvate of the compound. Those skilled in the art of organic chemistry and/or medicinal chemistry will appreciate that many organic compounds can form complexes with solvents in which they are reacted or from which they are precipitated or crystallised. Such complexes are referred to as “solvates”, and as used herein, the term “solvate” refers to such a complex of solute (e.g., a compound, salt of a compound) and solvent. Examples of solvents that may form pharmaceutically acceptable solvates include, but are not limited to, isopropanol, ethanol, methanol, DMSO, ethylacetate, acetic acid, and ethanolamine. If the solvate is water, the solvate may be conventionally referred to as a “hydrate”. In some embodiments, the pharmaceutically acceptable solvate is a pharmaceutically acceptable hydrate. The hydrate may be, for example, a mono-hydrate, a di-hydrate, a tri-hydrate, etc. Unless otherwise specified herein, reference to a particular compound also includes solvates thereof.

### Prodrugs

It may be convenient or desirable to prepare, purify, and/or handle the compound in the form of a prodrug. The term “prodrug”, as used herein, pertains to compound which, when metabolised (e.g., in vivo), yields the desired active compound. Typically, the prodrug is inactive, or less active than the desired active compound, but may provide advantageous handling, administration, or metabolic properties.

Also, as would be understood by a person skilled in the art of organic chemistry and/or medicinal chemistry, some prodrugs are activated enzymatically to yield the active compound, or a compound which, upon further chemical reaction, yields the active compound. For example, the prodrug may be a sugar derivative or other glycoside conjugate, or may be an amino acid ester derivative.

### Dosages

The amount of active ingredient that is required to achieve a therapeutic effect will, of course, vary with the particular compound, the route of administration, the subject under treatment, including the type, species, age, weight, sex, and medical condition of the subject being treated, and the renal and hepatic function of the subject, and the particular condition, disorder or disease being treated, as well as its severity. An ordinary skilled physician or clinician can readily determine and prescribe the effective amount of the drug required to prevent or treat the condition, disorder or disease.

Dosages of a compound of Formulas I, II, III or IV, or pharmaceutically acceptable salts, hydrates, stereoisomers, or prodrugs thereof, when used for the indicated effects, may range between, for example, about 0.01 mg per kg of body weight (mg/kg) to about 1000 mg/kg. In one example, the dosage of a compound of Formulas I, II, III or IV, or pharmaceutically acceptable salts, hydrates, stereoisomers, or prodrugs thereof, is between about 0.01 and 1000, 0.1 and 500, 0.1 and 200, 1 and 200 mg/kg. In one example, the dosage of a compound of Formulas I, II, III, or IV, or pharmaceutically acceptable salts, hydrates, stereoisomers, or prodrugs thereof, is between about 0.01 and 1000 mg/kg. In one example, the dosage of a compound of Formulas I, II, III or IV, or pharmaceutically acceptable salts, hydrates, stereoisomers, or prodrugs thereof, is between about 0.1 and 200 mg/kg. In one example, the dosage of a compound of Formulas I, II, III, or IV, or pharmaceutically acceptable salt, hydrate, or prodrug thereof, is between about 1 and 200 mg/kg. In one example, the dosage of a compound of Formulas I, II, III, or IV, or pharmaceutically acceptable salt, hydrate, or prodrug thereof, is greater than about 0.01, 0.1, 1, 10, 20, 50, 75, 100, 500, 1000 mg/kg. In one example, the dosage of a compound of Formulas I, II, III, or IV, or pharmaceutically acceptable salt, hydrate, stereoisomer, or prodrug thereof, is less than about 5000, 1000, 75, 50, 20, 10, 1, 0.1 mg/kg.

A compound of Formulas I, II, III, or IV, or pharmaceutically acceptable salt, hydrate, stereoisomer or prodrug thereof, may for example be administered as a single daily dose, or otherwise the total daily dosage may be administered in divided doses of two, three, or four times daily. In one example, the compound of Formulas I, II, III, or IV, or pharmaceutically acceptable salt, hydrate, or stereoisomer thereof, may be dosed less frequently than once per day, for example once per two days, three days, four days, five days, six days, or once per week.

Accordingly, dosages of a compound of Formulas I, II, III, or IV, or pharmaceutically acceptable salt, hydrate, stereoisomer, or prodrug thereof, when used for the indicated effects, may range between, for example, about 0.01 mg per kg of body weight per day (mg/kg/day) to about and including 1000 mg/kg/day. In one example, the dosage of a compound of Formulas I, II, III, or IV, or pharmaceutically acceptable salt, hydrate, stereoisomer, or prodrug thereof, is between about 0.01 and including 1000, or between 0.1 and including 500, or 0.1 and including 200, or 1 and including 200 mg/kg/day. In one example, the dosage of a compound of Formulas I, II, III, or IV, or pharmaceutically acceptable salt, hydrate, stereoisomer, or prodrug thereof, is between about 0.01 and including 1000 mg/kg/day. In one example, the dosage of a compound of Formula I, or pharmaceutically acceptable salt, hydrate, or prodrug thereof, is between about 0.1 and including 200 mg/kg/day, or between about 0.001 mg/kg to and including about 600 mg/kg. In one example, the dosage of a compound of Formulas I, II, III, or IV, or pharmaceutically acceptable salt, hydrate, stereoisomer, or prodrug thereof, is between about 1 and including 200 mg/kg/day. In one example, the dosage of a compound of Formula I, or pharmaceutically acceptable salt, hydrate, stereoisomer, or prodrug thereof, is greater than about 0.01, 0.1, 1, 10, 20, 50, 75, 100, 500, 1000 mg/kg/day. In one example, the dosage of a compound of Formulas I, II, III, or IV, or pharmaceutically acceptable salt, hydrate, stereoisomer, or prodrug thereof, is less than about 5000, 1000, 75, 50, 20, 10, 1, 0.1 or mg/kg/day, respectively.

It should be understood that the doses above are suitable for compounds of Formulas I, II, III and IV.

### Combination Therapy

Whilst a compound of Formulas I, II, III, or IV, or pharmaceutically acceptable salt, hydrate, or prodrug thereof, may be used as the sole active agent in a medicament, it is also possible for a compound of Formulas I, II, III, or IV, or pharmaceutically acceptable salt, hydrate, or prodrug thereof, to be used in combination with one or more further therapeutic agents. Accordingly, in one example, a compound of Formula I, or pharmaceutically acceptable salt, hydrate, stereoisomer, or prodrug thereof, is used in combination with one or more further therapeutic agents. The present disclosure therefore also provides a combination of a compound of Formulas I, II, III, or IV, or pharmaceutically acceptable salt, hydrate, or prodrug thereof, and a further therapeutic agent. The present disclosure also provides a pharmaceutical composition comprising a combination of a compound of Formulas I, II, III, or IV, or pharmaceutically acceptable salt, hydrate, or prodrug thereof, a further therapeutic agent, and a pharmaceutically acceptable excipient. Such one or more further therapeutic agents may for example be recombinant human growth hormone (rhGH), human growth hormone, recombinant human IGF-1 (rhIGF-1), IGF-1, IGF-2, any IGF Binding Protein (IGFBP), IGFBP-3, insulin, any statin, any appetite suppressant, transforming growth factor-β1, activin, nerve growth factor, growth hormone binding protein, basic fibroblast growth factor, acidic fibroblast growth factor, the hst/Kfgk gene product, FGF-3, FGF-4, FGF-6, keratinocyte growth factor, androgen-induced growth factor, int-2, fibroblast growth factor, homologous factor-1 (FHF-1), FHF-2, FHF-3, FHF-4, keratinocyte growth factor 2, glial activating factor, FGF-10, FGF-16, ciliary neurotrophic factor, brain derived nerve growth factor, neurotrophin 3, neurotrophin 4, bone morphogenetic protein 2 (BMP-2), glial-cell line derived neurotrophic factor, activity-dependant neurotrophic factor, cytokine leukaemia inhibiting factor, oncostatin M, an interleukin, a-interferon, β-interferon, γ-interferon, consensus interferon, TNF-a, clomethiazole; kynurenic acid, Semax, tacrolimus, L-threo-1-phenyl-2-decanoylamino3, 3-morpholino-1-propanol, adrenocorticotropin-(4-9) analogue (ORG 2766), dizolcipine [MK-801], selegiline, NPS1506, GV1505260, MK-801, GV150526, 2,3-dihydroxy-6-nitro-7-sulfamoylbenzo(f)quinoxaline (NBQX), LY303070, LY300164, and the anti-MAdCAM-1 antibody MECA-367.

The compound of Formulas I, II, III, or IV, or pharmaceutically acceptable salt, hydrate, stereoisomer, or prodrug thereof, and the one or more further pharmaceutically active agents may be administered simultaneously, subsequently or separately. For example, they may be administered as part of the same composition, or by administration of separate compositions. In one example, the compound of Formulas I, II, III, or IV, or pharmaceutically acceptable salt, hydrate, stereoisomer, or prodrug thereof, is administered in combination with recombinant human growth hormone.

The further therapeutic agents, when employed in combination with a compound of Formulas I, II, III, or IV, or pharmaceutically acceptable salt, hydrate, stereoisomer, or prodrug thereof, may be used for example in those amounts indicated in the Prescribers' Digital Reference or as otherwise determined by one of ordinary skill in the art.

### Synthesis of Compounds of Formula I

Numerous synthetic routes to the compounds of Formulas I, II, III and IV can be devised by any person skilled in the art and the possible synthetic routes described below are not intended to be limiting. Where appropriate, any initially produced compound of Formula I can be converted into another compound of Formula I by known methods.

Starting materials and reagents used in preparing the compounds of Formula I are either available from commercial suppliers such as Aldrich Chemical Company (Milwaukee, Wis.), Bachem (Torrance, Calif.), Sigma (St.Louis, Mo.), or are prepared by methods well known to the person of ordinary skill in the art following procedures described in such references as Fieser and Fieser's Reagents for Organic Synthesis, vols 1-17, John Wiley and Sons, New York, N.Y., 1991; Rodd's Chemistry of Carbon Compounds, vols. 1-5 and supplements, Elsevier Science Publishers, 1989; Organic Reactions, vols. 1-40, John Wiley and Sons, New York, N.Y., 1991; March J; Advanced Organic Chemistry, 4th ed. John Wiley and Sons, New York, N.Y., 1992; and Larock: Comprehensive Organic Transformations, VCH Publishers, 1989. In most instances, amino acids and their esters or amides, and protected amino acids, are widely commercially available; and the preparation of modified amino acids and their amides or esters are extensively described in the chemical and biochemical literature and thus well-known to persons of ordinary skill in the art.

Starting materials, intermediates, and final products this disclosure may be isolated and purified using conventional techniques, including filtration, distillation, crystallization, chromatography, and the like. They may be characterized using conventional methods, including physical constants and spectral data.

Cyclic G-2-AllylP is a cyclic dipeptide (bicyclic 2,5-diketopiperazine), and is a member of the class of compounds known as cyclic GPs (“cGP”). In general, cGPs and cyclic G-2-AllylP may be prepared by methods such as are already well-known to persons of ordinary skill in the art of peptide and modified peptide synthesis, following the reaction schemes set forth herein, or by following other methods well-known to those of ordinary skill in the art of the synthesis of peptides and analogues. See for example, Bodanzsky: Principles of Peptide Synthesis, Berlin, New York: Springer-Verlag 1993.

Synthesis of the diketopiperazine compounds of this disclosure may be by solution phase synthesis or via the solid-phase synthesis method exemplified by Merrifield et al. 1963 J. Amer. Chem. Soc.: 85, 2149-2156. Solid phase synthesis may be performed using commercial peptide synthesizers, such as the Applied Biosystems Model 430A, using the protocols established for the instrument.

Specific examples of diketopiperazine synthesis can be found in Fischer, 2003, J. Peptide Science: 9: 9-35 and references therein. A person of ordinary skill in the art will have no difficulty, taking account of that skill and the knowledge available, and of this disclosure, in developing one or more suitable synthetic methods for compounds of this invention.

The choice of appropriate protecting groups for the method chosen (solid-phase or solution-phase), and of appropriate substrates if solid-phase synthesis is used, will be within the skill of a person of ordinary skill in the art. Appropriate protecting groups for peptide synthesis include t-butyloxycarbonyl (Boc), fluorenylmethyloxycarbonyl (Fmoc), Benzyl (Bzl), t-amyloxycarbonyl (Aoc), tosyl (Tos), benzyloxycarbonyl (Z or Cbz), o-bromobenzyloxycarbonyl (BrZ) and the like. Additional protecting groups are identified in Goodman M. (ed.), “Synthesis of Peptides and Peptidomimetics” in Methods of organic chemistry (Houben-Weyl) (Workbench Edition, E22a,b,c,d,e; 2004; Georg Thieme Verlag, Stuttgart, New York).

The choice of coupling agent for the method chosen will also be within the skill of a person of ordinary skill in the art. Suitable coupling agents include DCC (N,N′-Dicyclohexylcarbodiimide), Bop (Benzotriazole-1-yl-oxy-tris-(dimethylamino)-phosphonium hexafluorophosphate), PyBop (Benzotriazol-1-yloxytripyrrolidinophosphonium hexafluorophosphate), BopCl (bis(2-oxo-3-oxazolidinyl)phosphinic chloride), 2-Chloro-1,3-dimethylimidazolidinium hexafluorophosphate (CIP) and the like. Other compounds may be used in the synthesis e.g. to prevent racemisation, such as HOBt (N-Hydroxybenzotriazole) and HOAt (1-Hydroxy-7-azabenzotriazole).

### EXAMPLES

The present disclosure is further illustrated by the following examples. These examples are offered by way of illustration only and are not intended to limit the scope of the disclosure.

### General: Materials and Methods

Flash chromatography was performed using Scharlau 60 (40-60 μm mesh) silica gel. Analytical thin layer chromatography was carried out on 0.20 mm pre-coated silica gel plates (ALUGRAM® SIL G/UV254) and compounds visualized using UV fluorescence, or heating of plates dipped in potassium permanganate in alkaline solution.

Melting points in degrees Celsius (° C.) were determined on an Electrothermal® melting point apparatus and are uncorrected.

Optical rotations were measured at 20° C. on a Perkin Elmer 341 polarimeter using 10 cm path length cells and are given in units of 10⁻¹ degcm² g⁻¹. Samples were prepared in the solvent indicated at the concentration specified (measured in g/100 cm³). IR spectra were recorded on a Perkin Elmer Spectrum One FT-IR spectrometer. The samples were prepared as thin films on sodium chloride discs or as solids in potassium bromide discs. A broad signal indicated by br. The frequencies (u) as absorption maxima are given in wavenumbers (cm⁻¹).

NMR spectra were recorded on a Bruker AVANCE DRX400 (¹H, 400 MHz; ¹³C, 100 MHz) or a Bruker AVANCE 300 (¹H, 300 MHz; ¹³C, 75 MHz) spectrometer at ambient temperatures. For ¹H NMR data chemical shifts are described in parts per million downfield from SiMe₄ and are reported consecutively as position (δH), relative integral, multiplicity (s=singlet, d=doublet, t=triplet, dd=doublet of doublets, m=multiplet, br=broad), coupling constant (J/Hz) and assignment. For ¹³C NMR data, chemical shifts are described in parts per million relative to CDCl₃ and are reported consecutively as position (SC), degree of hybridization as determined by DEPT experiments, and assignment. ¹H NMR spectra were referenced internally using SiMe₄ (δ 0.00) or CDCl₃ (δ 7.26). ¹³C NMR spectra were referenced internally using CDCl₃ (δ 77.0). When two sets of peaks arise in the NMR spectra due to different conformations around the glycine-proline amide bond, the chemical shift for the minor cis conformer is marked with an asterisk (*).

Accurate mass measurements were recorded on a VG-70SE mass spectrometer.

Hexane and dichloromethane were distilled prior to use. Methanol was dried using magnesium turnings and iodine, and distilled under nitrogen. Triethylamine was dried over calcium hydride and distilled under nitrogen.

### Example 1: Synthesis of (8aS)-Methyl-hexahydropyrrolo[1,2-a]pyrazine-1,-dione (Cyclic G-2-MeP

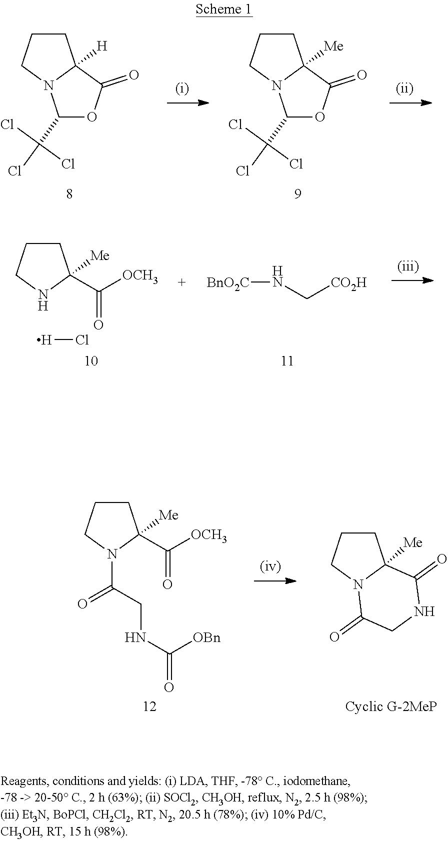

### (2R,5S)-4-Methyl-2-trichloromethyl-1-aza-3-oxabicyclo[3.3.0]octan-4-one (9)

n-BuLi (1.31 M, 4.68 cm³, 6.14 mmol) was added dropwise to a stirred solution of diisopropylamine (0.86 cm³, 6.14 mmol) in dry tetrahydrofuran (10 cm³) at −78° C. under an atmosphere of nitrogen. The solution was stirred for 5 min, warmed to 0° C. and stirred for 15 min. The solution was then added dropwise to a solution of oxazolidinone 8 (1.00 g, 4.09 mmol) in dry tetrahydrofuran (20 cm³) at −78° C. over 20 min (turned to a dark brown colour), stirred for a further 30 min then iodomethane (0.76 cm³, 12.3 mmol) was added dropwise over 5 min. The solution was warmed to −50° C. over 2 h. Water (15 cm³) was added and the solution warmed to room temperature and extracted with chloroform (3×40 cm³). The combined organic extracts were dried (MgSO₄), filtered and evaporated to dryness in vacuo to give a dark brown semi-solid. Purification of the residue by flash column chromatography (15% ethyl acetate-hexane) afforded oxazolidinone 9 (0.67 g, 63%) as a pale yellow solid: mp 55-57° C. (lit., 57-60° C.); δH (300 MHz, CDCl₃) 1.53 (3H, s, CH₃), 1.72-2.02 (3H, m, Proβ-H and Proγ-H₂), 2.18-2.26 (1H, m, Proβ-H), 3.15-3.22 (1H, m, Proδ-H), 3.35-3.44 (1H, m, Proδ-H) and 4.99 (1H, s, NCH).

### Methyl L-2-methylprolinate Hydrochloride (10)

### a) Using Acetyl Chloride

Oxazolidinone 9 (0.60 g, 2.33 mmol) was dissolved in dry methanol (15 cm³) under an atmosphere of nitrogen and acetyl chloride (0.33 cm³, 4.66 mmol) was added dropwise to the ice-cooled solution. The solution was heated under reflux for 4.5 h, then the solvent removed under reduced pressure to give a brown oil which was purified by flash column chromatography (10% CH₃OH—CH₂Cl₂) affording the hydrochloride 10 (0.2 g, 48%) as a flaky white solid: mp 107-109° C. (lit., 106-108° C.); δH (300 MHz, CDCl₃) 1.81 (3H, s, CH₃), 1.93-2.14 (3H, m, Proβ-H_{A}H_{B} and Proγ-H₂), 2.33-2.39 (1H, m, Proβ-H_{A}H_{B}), 3.52-3.56 (2H, m, Proδ-H₂) and 3.82 (3H, s, CO₂CH₃).

### b) Using Thionyl Chloride

An ice-cooled solution of oxazolidinone 9 (53 mg, 0.21 mmol) in dry methanol (1 cm³) was treated dropwise with thionyl chloride (0.045 cm³, 0.62 mmol). The solution was heated under reflux for 2.5 h, cooled and the solvent removed under reduced pressure to yield a brown oil. The oil was dissolved in toluene (5 cm3), concentrated to dryness to remove residual thionyl chloride and methanol then purified by flash column chromatography (10% CH₃OH—CH₂Cl₂) to afford the hydrochloride 10 (16 mg, 43%) as a flaky white solid. The ¹H NMR assignments were in agreement with those reported above.

### Methyl-N-benzyloxycarbonyl-glycyl-L-2-methylprolinate (12)

Dry triethylamine (0.27 cm³, 1.96 mmol) was added dropwise to a solution of hydrochloride 10 (0.11 g, 0.61 mmol) and N-benzyloxycarbonyl-glycine 11 (98.5%) (0.17 g, 0.79 mmol) in dry dichloromethane (35 cm³) under an atmosphere of nitrogen at room temperature, and the reaction mixture stirred for 10 min. Bis(2-oxo-3-oxazolidinyl)phosphinic chloride (BoPCl, 97%) (0.196 g, 0.77 mmol) was added and the resultant colourless solution was stirred for 20.5 h. The solution was washed successively with 10% aqueous hydrochloric acid (30 cm³) and saturated aqueous sodium hydrogen carbonate (30 cm³), dried (MgSO₄), filtered and evaporated to dryness in vacuo. Purification of the resultant residue by flash column chromatography (50-80% ethyl acetate-hexane; gradient elution) yielded dipeptide 12 (0.18 g, 92%) as a colourless oil. Amide 12 was shown to exist as a 98:2 trans:cis mixture of conformers by ¹³C NMR analysis (the ratio was estimated from the relative intensities of the resonances at δ 20.8 and 23.5 assigned to the Proγ-C atoms of the minor and major conformers, respectively): [α]_{D}−33.0 (c 1.0 in MeOH); nmax (film)/cm⁻¹ 3406, 2952, 1732, 1651, 1521, 1434, 1373, 1329, 1310, 1284, 1257, 1220, 1195, 1172, 1135, 1107, 1082, 1052, 1029, 986, 965, 907, 876, 829, 775, 738 and 699; δH (300 MHz, CDCl₃) 1.49 (3H, s, CH₃), 1.77-2.11 (4H, m, Proβ-H₂ and Proγ-H₂), 3.43-3.48 (2H, m, Proδ-H₂), 3.61 (3H, s, OCH₃), 3.85-3.89 (2H, m, Glyα-H₂), 5.04 (2H, s, PhCH₂), 5.76 (1H, br s, N—H) and 7.21-7.28 (5H, s, ArH); δC (75 MHz, CDCl₃) 13.8* (CH₃, Proα-CH₃), 21.1 (CH₃, Proα-CH₃), 20.8* (CH₂, Proγ-C), 23.5 (CH₂, Proγ-C), 38.0 (CH₂, Proβ-C), 40.8* (CH₂, Proβ-C), 43.3 (CH₂, Glyα-C), 45.5* (CH₂, Glyα-C), 46.6 (CH₂, Proδ-C), 48.7* (CH₂, Proδ-C), 51.9* (CH₃, OCH₃), 52.1 (CH₃, OCH₃), 60.0* (quat., Proα-C), 66.0 (quat., Proα-C), 66.3 (CH₂, PhCH₂), 68.6* (CH₂, PhCH₂), 127.5 (CH, Ph), 127.6 (CH, Ph), 127.9* (CH, Ph), 128.1 (CH, Ph), 128.3* (CH, Ph), 136.2 (quat., Ph), 155.9 (quat., NCO₂), 166.0 (quat., Gly-CON), 169.4* (quat., Gly-CON) and 173.6 (quat., CO₂CH₃); m/z (EI+) 334.1535 (M+. C₁₇H₂₂N₂O₅ requires 334.1529).

### (8aS)-Methyl-hexahydropyrrolo[1,2-a]pyrazine-1,4-dione (Cyclic G-2MeP)

To a solution of dipeptide 12 (0.167 g, 0.51 mmol) in methanol (8.0 cm³) was added 10% Pd on activated charcoal (8.1 mg, 0.076 mmol) and the vessel flushed with hydrogen gas. The resulting suspension was stirred vigorously under an atmosphere of hydrogen for 15 h. The mixture was then filtered through a Celite pad then a short plug of silica gel with methanol, and the solvent removed under reduced pressure to produce cyclic G-2MeP (83 mg, 98%) as a yellow solid: mp 133-135° C.; [α]_{D}−128.1 (c 0.52 in MeOH); δH (300 MHz, CDCl₃) 1.36 (3H, s, CH₃), 1.87-2.01 (3H, m, Proβ-H_{A}H_{B} and Proγ-H₂), 2.07-2.21 (1H, m, Proβ-H_{A}H_{B}), 3.45-3.64 (2H, m, Proδ-H₂), 3.82 (1H, dd, J 17.1 and 4.1, CH_{A}H_{B}NH), 3.99 (1H, d, J 17.1, CH_{A}H_{B}NH) and 7.66 (1H, br s, N—H); δC (75 MHz, CDCl₃) 20.2 (CH₂, Proγ-C), 23.2 (CH₃, Proα-CH₃), 35.0 (CH₂, Proβ-C), 44.7 (CH₂, Proδ-C), 45.9 (CH₂, CH₂NH), 63.8 (quat., Proα-C), 163.3 (quat., NCO) and 173.3 (quat., CONH); m/5 z (EI+) 168.08986 (M+. C₈H₁₂N₂O₂ requires 168.08988).

### Example 2: Synthesis of (8aS)-Methyl-spiro[cyclohexane-1,3(4H)-tetrahydropyrrolo[1,2-a]pyrazine]-1,4(2H)-dione (Cyclic cyclohexyl-G-2-MeP) Formula IV

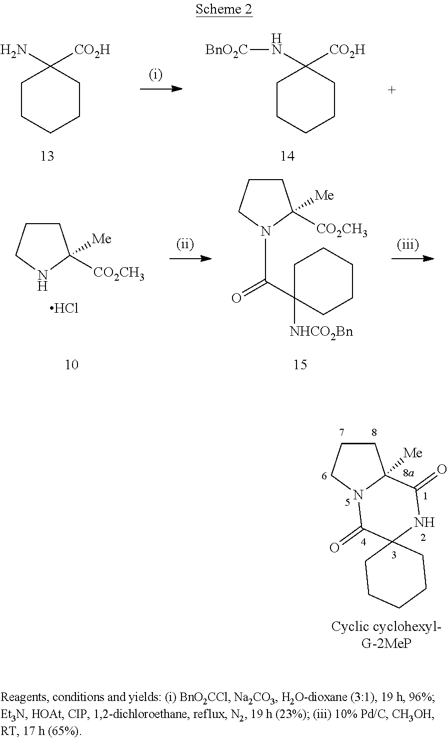

### N-benzyloxycarbonyl-1-aminocyclohexane-1-carboxylic Acid (14)

To a suspension of 1-aminocyclohexanecarboxylic acid 13 (0.72 g, 5.02 mmol) and sodium carbonate (1.6 g, 15.1 mmol) were dissolved in water-dioxane (21 cm³, 3:1) was added benzyl chloroformate (0.79 cm³, 5.52 mmol) was added dropwise and the solution was stirred at room temperature for 19.5 h. The aqueous layer was washed with diethyl ether (60 cm³), acidified with 2 M HCl and extracted with ethyl acetate (2×60 cm³). The organic layers were combined, dried (MgSO₄), filtered and evaporated under reduced pressure to produce a colourless oil, which solidified on standing to crude carbamate 14 (1.23 g, 88%) as a white solid: mp 152-154° C. (lit., 148-150° C.); δH (400 MHz, CDCl₃) 1.27-1.56 (3H, m, 3×cyclohexyl-H), 1.59-1.73 (3H, m, 3×cyclohexyl-H), 1.85-1.91 (2H, m, 2×cyclopentyl-H), 2.05-2.09 (2H, m, 2×cyclopentyl-H), 5.02 (1H, br s, N—H), 5.12 (2H, s, OCH₂Ph) and 7.27-7.36 (5H, s, Ph); δC (100 MHz, CDCl₃) 21.1 (CH₂, 2×cyclohexyl-C), 25.1 (CH₂, 2×cyclohexyl-C), 32.3 (CH₂, cyclohexyl-C), 5 59.0 (quat.,1-C), 67.1 (CH₂, OCH₂Ph), 128.1 (CH, Ph), 128.2 (CH, Ph), 128.5 (CH, Ph), 136.1 (quat., Ph), 155.7 (quat., NCO2) and 178.7 (quat., CO₂H).

### Methyl-N-benzyloxycarbonyl-cyclohexyl-glycyl-L-2-methylprolinate (15)

Dry triethylamine (0.21 cm³, 1.5 mmol) was added dropwise to a solution of hydrochloride 10 (84.0 mg, 0.47 mmol), carboxylic acid 14 (0.17 g, 0.61 mmol) and 1-hydroxy-7-azabenzotriazole (16 mg, 0.12 mmol) in dry 1,2-dichloroethane (26 cm³) under an atmosphere of nitrogen at room temperature, and the reaction mixture stirred for 10 min. 2-Chloro-1,3-dimethylimidazolidinium hexafluorophosphate (0.13 g, 0.47 mmol) was added and the resultant solution heated under reflux for 21 h, then washed successively with 10% aqueous hydrochloric acid (30 cm³) and saturated aqueous sodium hydrogen carbonate (30 cm³), dried (MgSO₄), filtered and evaporated to dryness in vacuo. Purification of the resultant residue by flash column chromatography (40-50% ethyl acetate-hexane; gradient elution) yielded amide 15 (16 mg, 9%) as a white solid. Amide 15 was shown to exist as a 11:1 trans:cis mixture of conformers by ¹³C NMR analysis (the ratio was estimated from the relative intensities of the resonances at δ 41.3 and 48.2 assigned to the Proδ-C atoms of the minor and major conformers, respectively): mp 219-222° C.; [α]_{D}−44.9 (c 1.31 in CH₂Cl₂); nmax (film)/cm⁻¹ 3239, 2927, 1736, 1707, 1617, 1530, 1450, 1403, 1371, 1281, 1241, 1208, 1194, 1165, 1150, 1132, 1089, 1071, 1028, 984, 912, 25 796, 749, 739 and 699; δH (400 MHz, CDCl₃) 1.24-2.10 (17H, m, Proα-CH₃, Proβ-H₂, Proγ-H₂ and 5×cyclohexyl-H₂), 3.25-3.48 (1H, br m, Prod-H_{A}H_{B}), 3.61-3.87 (4H, br m, OCH₃ and Proδ-H_{A}H_{B}), 4.92-5.19 (3H, m, N—H and OCH₂Ph) and 7.35-7.37 (5H, s, Ph); δC (100 MHz, CDCl₃) 21.26 (CH₂, cyclohexyl-C), 21.33 (CH₂, cyclohexyl-C), 21.7 (CH₃, Proα-CH₃), 24.8 (CH₂, cyclohexyl-C), 25.0 (CH₂, Proγ-C), 29.4* (CH₂, cyclohexyl-C), 29.7* (CH₂, cyclohexyl-C), 31.1 (CH₂, cyclohexyl-C), 31.6 (CH₂, cyclohexyl-C), 31.9* (CH₂, cyclohexyl-C), 32.2* (CH₂, cyclohexyl-C), 32.8* (CH₂, cyclohexyl-C), 37.3 (CH₂, Proβ-C), 41.4* (CH₂, Proδ-C), 48.2 (CH₂, Proδ-C), 52.1 (CH₃, OCH₃), 59.1 (quat., Glyα-C), 66.7 (CH₂, OCH₂Ph), 67.3* (CH₂, OCH₂Ph), 67.4 (quat., Proα-C), 128.0* (CH, Ph), 128.1* (CH, Ph), 128.3 (CH, Ph), 128.5 (CH, Ph), 128.7 (CH, Ph), 136.6 (quat., Ph), 153.7 (quat., NCO₂), 171.0 (quat., Gly-CO) and 174.8 (quat., CO₂CH₃); m/z (EI+) 402.2151 (M⁺ C₂₂H₃₀N₂O₅ requires 402.2155).

### (8aS)-Methyl-spiro[cyclohexane-1,3(4H)-tetrahydropyrrolo[1,2-a]pyrazine]-1,4(2H)-dione (Cyclic Cyclohexyl-G-2MeP)

To a solution of amide 15 (40 mg, 0.01 mmol) in methanol (3.3 cm³) was added 10% Pd on activated charcoal (1.6 mg, 0.015 mmol) and the vessel flushed with hydrogen gas. The resulting suspension was stirred vigorously under an atmosphere of hydrogen for 61.5 h, then filtered through a Celite™ pad with methanol (15 cm³). The filtrate was concentrated to dryness under reduced pressure to produce a yellow semi-solid which was purified by reverse-phase C18 flash column chromatography (0-10% CH₃CN/H₂O; gradient elution) to produce cyclic cyclohexyl-G-2MeP (19 mg, 81%) as a white solid: mp 174-177° C.; [α]_{D}−63.8 (c 1.13 in CH₂Cl₂); nmax (film)/cm⁻¹ 3215, 2925, 2854, 1667, 1646, 1463, 1427, 1276, 1232, 1171, 1085, 1014, 900, 868, 818, 783, 726 and 715; δH (400 MHz, CDCl₃) 1.31-1.89 (12H, m, 9×cyclohexyl-H and 8a-CH₃), 1.94-2.15 (4H, m, 7-H₂ and 8-H₂), 2.26 (1H, td, J 13.7 and 4.5, 1×cyclohexyl-H), 3.44-3.51 (1H, m, 6-H_{A}H_{B}), 3.79-3.86 (1H, m, 6-H_{A}H_{B}) and 6.40 (1H, br s, N—H); δC (100 MHz, CDCl₃) 19.5 (CH₂, 7-C), 20.6 (CH₂, cyclohexyl-C), 20.8 (CH₂, cyclohexyl-C), 24.5 (CH₂, cyclohexyl-C), 25.0 (CH₃, 8a-CH₃), 33.7 (CH₂, cyclohexyl-C), 36.3 (CH₂, 8-C), 36.5 (CH₂, cyclohexyl-C), 44.7 (CH₂, 6-C), 59.5 (quat., 8a-C), 64.0 (quat., 3-C), 168.1 (quat., 4-C) and 171.6 (quat., 1-C); m/z (EI⁺) 236.15246 (M⁺ C₁₃H₂₀N₂O₂ requires 236.15248).

### Example 3: Synthesis of (8aS)-Allyl-hexahydropyrrolo[1,2-a]pyrazine-1,4-dione (Cyclic G-2-AllylP)

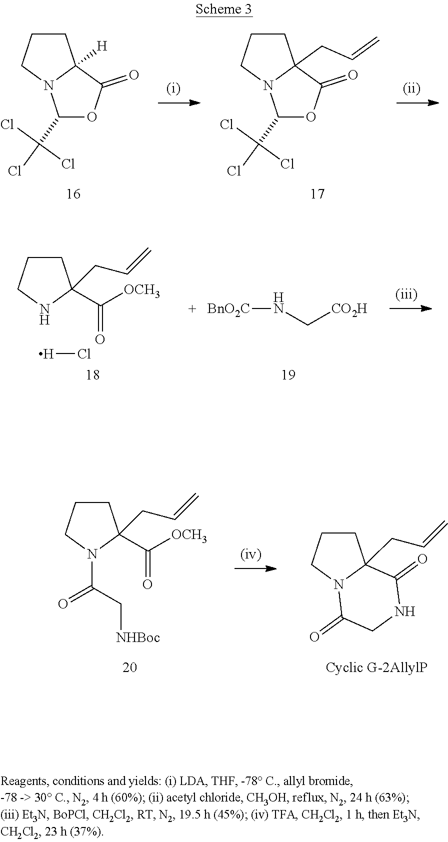

### (2R,5S)-4-Allyl-2-trichloromethyl-1-aza-3-oxabicyclo[3.3.0]octan-4-one (17)

n-BuLi (1.31 M, 9.93 cm³, 13.0 mmol) was added dropwise to a stirred solution of diisopropylamine (1.82 cm³, 13.0 mmol) in dry tetrahydrofuran (20 cm³) at −78° C. under an atmosphere of nitrogen. The solution was stirred for 5 min, warmed to 0° C., stirred for 15 min then added dropwise to a solution of pro-oxazolidinone 16 (2.12 g, 8.68 mmol) in dry tetrahydrofuran (40 cm³) at −78° C. over 20 min and the reaction mixture was stirred for a further 30 min then allyl bromide (2.25 cm³, 26.0 mmol) was added dropwise over 5 min. The solution was warmed slowly to −30° C. over 4 h, quenched with H₂O (30 cm³) and the mixture warmed to room temperature and extracted with chloroform (3×80 cm³). The combined organic extracts were dried (MgSO₄), filtered and evaporated to dryness in vacuo to produce a dark brown semi-solid which was purified by flash column chromatography (10-20% ethyl acetate-hexane; gradient elution) to produce oxazolidinone 17 (1.48 g, 60%) as an orange oil which solidified at 0° C., for which the nmr data were in agreement with that reported in the literature: δH (400 MHz, CDCl₃) 1.58-1.92 (2H, m, Proγ-H₂), 1.96-2.14 (2H, m, Proβ-H₂), 2.50-2.63 (2H, m, Proδ-H₂), 3.12-3.23 (2H, m, CH₂—CH═CH₂), 4.97 (1H, s, NCH), 5.13-5.18 (2H, m, CH═CH₂) and 5.82-5.92 (1H, m, CH═CH₂); δC (100 MHz, CDCl₃) 25.1 (CH₂, Proγ-C), 35.1 (CH₂, Proβ-C), 41.5 (CH₂, Proδ-C), 58.3 (CH₂, CH₂CH═CH₂), 71.2 (quat., Proα-C), 100.4 (quat., CCl₃), 102.3 (CH, NCH), 119.8 (CH₂, CH₂CH═CH₂), 131.9 (CH, CH₂CH═CH₂) and 176.1 (quat., C═O); m/z (CI+) 284.0009 [(M+H)⁺. C₁₀H₁₃C₁₃NO₂ requires 284.0012], 285.9980 [(M+H)⁺. C₁₀H₁₃³⁵Cl³⁷Cl₃NO₂ requires 285.9982], 287.9951 [(M+H)⁺. C₁₀H₁₃³⁵Cl³⁷Cl₂NO₂ requires 287.9953] and 289.9932 [(M+H)⁺. C₁₀H₁₃³⁷Cl₃NO₂ requires 289.9923].

### Methyl L-2-allylprolinate Hydrochloride (18)

An ice-cooled solution of oxazolidinone 17 (0.64 g, 2.24 mmol) in dry methanol (15 cm³) was treated dropwise with a solution of acetyl chloride (0.36 cm³, 5.0 mmol) in methanol (5 cm³). The solution was heated under reflux for 24 h, then cooled and the solvent removed under reduced pressure. The resultant brown oil was dissolved in toluene (40 cm³) and concentrated to dryness to remove residual thionyl chloride and methanol, then purified by flash column chromatography (5-10% CH₃OH—CH₂Cl₂; gradient elution) to afford hydrochloride 18 (0.29 g, 63%) as a green solid for which the NMR data were in agreement with that reported in the literature: δH (300 MHz, CDCl₃) 1.72-2.25 (3H, m, Proβ-H_{A}H_{B} and Proγ-H₂), 2.32-2.52 (1H, m, Proβ-H_{A}H_{B}), 2.72-3.10 (2H, m, Proδ-H₂), 3.31-3.78 (2H, m, CH₂CH═CH₂), 3.84 (3H, s, CO₂CH₃), 5.20-5.33 (2H, m, CH═CH₂), 5.75-5.98 (1H, m, CH═CH₂) and 8.06 (1H, br s, N—H); m/z (CI+) 170.1183 [(M+H)⁺. C₉H₁₆NO₂ requires 170.1181].

### Methyl-N-tert-butyloxycarbonyl-glycyl-L-2-allylprolinate (20)

Dry triethylamine (0.28 cm³, 2.02 mmol) was added dropwise to a solution of hydrochloride 18 (0.13 g, 0.63 mmol) and N-tert-butyloxycarbonyl-glycine 19 (0.14 g, 0.82 mmol) in dry dichloromethane (35 cm³) under an atmosphere of nitrogen at room temperature, and the reaction mixture was stirred for 10 min. Bis(2-oxo-3-oxazolidinyl)phosphinic chloride (BoPCl, 97%) (0.20 g, 0.80 mmol) was added and the solution stirred for 19.5 h, then washed successively with 10% aqueous hydrochloric acid (35 cm³) and saturated aqueous sodium hydrogen carbonate (35 cm³), dried (MgSO₄), filtered and evaporated to dryness in vacuo. Purification of the resultant residue by flash column chromatography (40% ethyl acetate-hexane) yielded dipeptide 20 (0.09 g, 45%) as a light yellow oil: [α]_{D}+33.8 (c 0.83 in CH₂Cl₂); nmax (film)/cm⁻¹ 3419, 5 3075, 2977, 2930, 2874, 1739, 1715, 1656, 1499, 1434, 1392, 1366, 1332, 1268, 1248, 1212, 1168, 1122, 1051, 1026, 1003, 943, 919, 867, 830, 779, 739, 699 and 679; δH (300 MHz, CDCl₃) 1.42 [9H, s, C(CH₃)₃], 1.93-2.08 (4H, m, Proβ-H₂ and Proγ-H₂), 2.59-2.67 (1H, m, CH_{A}H_{B}CH═CH₂), 3.09-3.16 (1H, m, CH_{A}H_{B}CH═CH₂), 3.35-3.44 (1H, m, Proδ-H_{A}H_{B}), 3.56-3.62 (1H, m, Proδ-H_{A}H_{B}), 3.70 (3H, s, OCH₃), 3.89 (2H, d, J 4.2, Glyα-H₂), 5.06-5.11 (2H, m, CH═CH₂), 5.42 (1H, br s, Gly-NH) and 5.58-5.72 (1H, m, CH═CH₂); δC (75 MHz, CDCl₃) 23.7 (CH₂, Proγ-C), 28.3 [CH₃, C(CH₃)₃], 35.0 (CH₂, Proβ-C), 37.6 (CH₂, CH₂CH═CH₂), 43.3 (CH₂, Glyα-C), 47.5 (CH₂, Proδ-C), 52.5 (CH₃, OCH₃), 68.8 (quat., Proα-C), 79.5 [quat., C(CH₃)₃], 119.4 (CH₂, CH═CH₂), 132.9 (CH, CH═CH₂), 155.7 (quat., NCO₂), 166.9 (quat., Gly-CON) and 173.8 (quat., CO₂CH₃); m/z (EI+) 326.1845 (M⁺. C₁₆H₂₆N₂O₅ requires 326.1842).

### (8aS)-Allyl-hexahydropyrrolo[1,2-a]pyrazine-1,4-dione (Cyclic G-2AllylP) Formula II

To a solution of dipeptide 20 (0.09 g, 0.28 mmol) in dichloromethane (9 cm³) at room temperature was added trifluoroacetic acid (1 cm³, 0.013 mmol) dropwise and the reaction mixture was stirred for 1 h under an atmosphere of nitrogen. The solution was evaporated under reduced pressure to give a colorless oil which was dissolved in dichloromethane (10 cm³), dry triethylamine (0.096 cm³, 0.69 mmol) was added and the reaction mixture stirred for 4.5 h, after which further triethylamine (0.096 cm³, 0.69 mmol) was added. The reaction mixture was stirred overnight, concentrated to dryness to give a green oil which was purified by flash column chromatography (10% CH₃OH—CH₂Cl₂) to produce cyclic G-2AllylP (20 mg, 37%) as an off-white solid: mp 106-109° C.; [α]_{D}−102.7 (c 0.95 in CH₂Cl₂); nmax (CH₂Cl₂)/cm−1 3456, 3226, 2920, 1666, 1454, 1325, 1306, 1299, 1210, 1133, 1109, 1028, 1010, 949, 928, 882, 793, 761 and 733; δH (400 MHz, CDCl₃) 1.92-2.01 (2H, m, Proγ-H₂), 2.09-2.16 (2H, m, Proβ-H₂), 2.39-2.56 (2H, m, CH₂CH₂═CH₂), 3.46-3.53 (1H, m, Proδ-H_{A}H_{B}), 3.78-3.87 (2H, m, Proδ-H_{A}H_{B} and Glyα-H_{A}H_{B}), 4.09 (1H, d, J 17.2, Glya-HAHB), 5.16-5.20 (2H, m, CH═CH2), 5.73-5.84 (1H, m, CH═CH2) and 7.17 (1H, br s, N—H); δC (100 MHz, CDCl₃) 20.1 (CH₂, Proγ-C), 34.1 (CH₂, Proβ-C), 41.7 (CH₂, CH₂CH₂═CH₂), 44.9 (CH₂, Proδ-C), 46.4 (CH₂, Glyα-C), 67.2 (quat., Proα-C), 120.9 (CH₂, CH═CH₂), 131.0 (CH, CH═CH₂), 163.4 (quat., NCO) and 171.7 (quat., CONH); m/z (EI+) 195.1132 (M⁺. C₁₀H₁₅N₂O₂ requires 5 195.1134).

### Example 4: Synthesis of (8aS)-Methyl-spiro[cyclopentane-1,3(4H)-tetrahydropyrrolo[1,2-a]pyrazine]-1,4(2H)-dione (Cyclic Cyclopentyl-G-2-MeP) Formula III

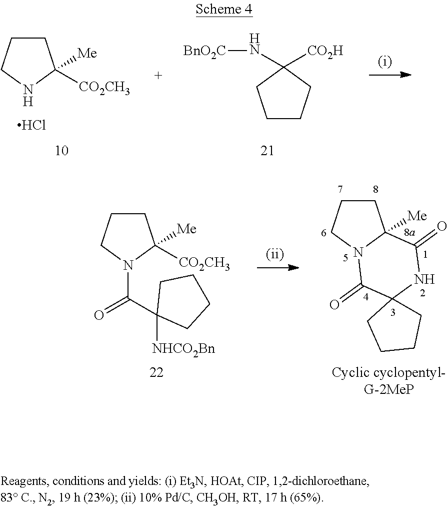

### N-Benzyloxycarbonyl-1-aminocyclopentane-1-carboxylic Acid (21)

A solution of benzyl chloroformate (0.29 g, 1.1 mmol) in dioxane 5 (2.5 cm³) was added dropwise to a solution of 1-aminocyclopentanecarboxylic acid (Fluka) (0.20 g, 1.54 mmol) and sodium carbonate (0.49 g, 4.64 mmol) in water (5 cm³) at 0° C. Stirring was continued at room temperature overnight and the reaction mixture washed with ether. The aqueous layer was acidified with 2M hydrochloric acid, extracted with ethyl acetate, dried (Na₂SO₄), filtered and the solvent removed to afford carbamate 21 (0.253 g, 62%) as an oil which solidified on standing. Carbamate 21 was shown to be a 70:30 mixture of conformers by ¹H NMR analysis (the ratio was estimated from the integration of the resonances at δ 5.31 and 7.29-7.40, assigned to the N—H protons of the major and minor conformers, respectively): mp 70-80° C. (lit. 1 82-86° C., ethyl acetate, petroleum ether); δH (400 MHz; CDCl₃; Me₄Si) 1.83 (4H, br s, 2×cyclopentyl-H₂), 2.04 (2H, br s, cyclopentyl-H₂), 2.20-2.40 (2H, m, cyclopentyl-H₂), 5.13 (2H, br s, OCH₂Ph), 5.31 (0.7H, br s, N—H) and 7.29-7.40 (5.3H, m, Ph and N—H*); δC (100 MHz; CDCl₃) 24.6 (CH₂, cyclopentyl-C), 37.5 (CH₂, cyclopentyl-C), 66.0 (quat., cyclopentyl-C), 66.8 (CH₂, OCH₂Ph), 128.0 (CH, Ph), 128.1 (CH, Ph), 128.4 (CH, Ph), 136.1 (quat, Ph), 155.8 (quat., NCO₂) and 179.5 (quat., CO₂H).

### Methyl N-benzyloxycarbonyl cyclopentyl-glycyl-L-2-methylprolinate (22)

Dry triethylamine (0.19 cm³, 1.4 mmol) was added dropwise to a solution of hydrochloride 10 (78 mg, 0.43 mmol), carboxylic acid 21 (0.15 g, 0.56 mmol) and 1-hydroxy-7-azabenzotriazole (Acros) (15 mg, 0.11 mmol) in dry 1,2-dichloroethane (24 cm³) under an atmosphere of nitrogen at room temperature, and the reaction mixture stirred for 10 min. 2-Chloro-1,3-dimethylimidazolidinium hexafluorophosphate (CIP) (Aldrich) (0.12 g, 0.43 mmol) was added and the resultant solution heated under reflux for 19 h, then washed successively with 10% aqueous hydrochloric acid (30 cm³) and saturated aqueous sodium hydrogen carbonate (30 cm³), dried (MgSO₄), filtered and evaporated to dryness in vacuo. Purification of the resultant residue by flash column chromatography (60% ethyl acetate-hexane) yielded amide 22 (39 mg, 23%) as a white solid. Amide 22 was shown to exist as a 3:1 trans:cis mixture of carbamate conformers by ¹³C NMR analysis (the ratio was estimated from the relative intensities of the resonances at δ 154.1 and 155.7 assigned to the carbamate carbonyl-C atoms of the major and minor conformers, respectively): mp 200-203° C.; [α]_{D}−54.5 (c 1.52 in CH₂Cl₂); nmax (film)/cm⁻¹ 3432, 3239, 5 3042, 2953, 1736, 1712, 1627, 1540, 1455, 1417, 1439, 1374, 1282, 1256, 1216, 1194, 1171, 1156, 1136, 1100, 1081, 1042, 1020, 107, 953, 917, 876, 756 and 701; δH (400 MHz, CDCl₃) 1.33-1.53 (3H, br m, Proα-CH₃), 1.62-2.20 (11H, m, Proβ-H₂, Proγ-H₂ and 7×cyclopentyl-H), 2.59-2.71 (1H, br m, 1×cyclopentyl-H), 3.31-3.42 (1H, br m, Prod-H_{A}H_{B}), 3.58-3.79 (4H, br m, OCH₃ and Proδ-H_{A}H_{B}), 4.92-5.17 (3H, m, N—H and OCH₂Ph) and 7.27-7.42 (5H, s, Ph); δC (100 MHz, CDCl₃) 21.7 (CH₃, Proα-CH₃), 24.1* (CH₂, cyclopentyl-C), 24.2 (CH₂, cyclopentyl-C), 24.4 (CH₂, Proγ-C), 24.5 (CH₂, cyclopentyl-C), 36.4 (CH₂, cyclopentyl-C), 37.1 (CH₂, cyclopentyl-C), 37.2* (CH₂, cyclopentyl-C), 37.7 (CH₂, Proβ-C), 38.2* (CH₂, cyclopentyl-C), 48.5 (CH₂, Proδ-C), 52.1 (CH₃, OCH₃), 66.6 (CH₂, OCH₂Ph), 66.9 (quat., Proα-C), 67.2 (quat., Glyα-C), 127.8 (CH, Ph), 128.2 (CH, Ph), 128.4 (CH, Ph), 136.6 (quat., Ph), 154.1 (quat., NCO₂), 155.7* (quat., NCO₂), 170.5 (quat., Gly-CO) and 174.7 (quat., CO₂CH₃); m/z (EI+) 388.1991 (M⁺. C₂₁H₂₈N₂O₅ requires 388.1998).

### (8aS)-Methyl-spiro[cyclopentane-1,3(4H)-tetrahydropyrrolo[1,2-a]pyrazine]-1,4(2H)-dione (Cyclic cyclopentyl-G-2MeP) Formula III

To a solution of amide 22 (54 mg, 0.14 mmol) in methanol (4.6 cm³) was added 10% Pd on activated charcoal (2.2 mg, 0.021 mmol) and the vessel flushed with hydrogen gas. The resulting suspension was stirred vigorously under an atmosphere of hydrogen for 17 h, then filtered through a Celite™ pad with methanol (15 cm³). The filtrate was concentrated to dryness under reduced pressure to give a yellow semi-solid which was purified by reverse-phase C18 flash column chromatography (0-10% CH₃CN/H₂O; gradient elution) to afford cyclic cyclopentyl-G-2MeP (20 mg, 65%) as a yellow solid: mp 160-163° C.; [α]_{D}−97.9 (c 1.61 in CH₂Cl₂); nmax (film)/cm⁻¹ 3429, 2956, 2928, 2856, 1667, 1643, 1463, 1432, 1373, 1339, 1254, 1224, 1175, 1086, 1048, 976, 835, 774 and 730; δH (300 MHz, CDCl₃) 1.47 (3H, br s, 8a-CH₃), 1.56-2.19 (11H, m, 8-H₂, 7-H₂ and 7×cyclopentyl), 2.58-2.67 (1H, br m, 1×cyclopentyl), 3.48-3.56 (1H, m, 6-HAHB), 3.72-3.82 (1H, m, 6-H_{A}H_{B}) and 6.56 (1H, br s, N—H); δC (75 MHz, CDCl₃) 19.9 (CH₂, 7-C), 24.6 (CH₂, cyclopentyl), 24.92 (CH₃, 8a-CH₃), 24.93 (CH₂, cyclopentyl), 36.0 (CH₂, 8-C), 38.7 (CH₂, cyclopentyl), 41.9 (CH₂, cyclopentyl), 44.8 (CH₂, 6-C), 64.3 (quat., 8a-C), 66.8 (quat., 3-C), 168.3 (quat., 4-C) and 172.2 (quat., 1-C); m/z (EI+) 222.1369 (M⁺. C₁₂H₁₈N₂O₂ requires 222.1368).

### Animal Model and Clinical Studies

The following pharmacological studies described below demonstrate efficacy of cyclic G-2-AllylP in attenuation of symptoms of Prader-Willi Syndrome (PWS). They are not intended to be limiting, and other compositions and methods of this invention can be developed without undue experimentation. All of those compositions and methods are considered to be part of this disclosure. All the following experiments were carried out using protocols developed under guidelines approved by the University of Chile Animal Ethics Committee or comparable regulatory bodies.

### Example 5: Delivery of cG2-AllylP Into the Brain After Oral Administration

In an in vivo study, male Sprague Dawley rats (aged 14 weeks) received a single dose of cG-2-AllylP, either 100 mg/kg or 200 mg/kg by oral gavage. Cerebrospinal fluid (CSF) and whole blood were collected at 1.5 and 4 hours post-dose, and brain tissue was collected at 4 hours post-dose to evaluate cG-2-AllylP exposure. Table 1 below shows the blood, CSF, and brain cG-2-AllylP in CSF and blood 1.5 hours after dosing.

TABLE 1
Concentration of cG-2-AllylP in CSF,
Blood and the Brain in Wild Type Rats

| | Mean Test Article Exposure | | |
|---|---|---|---|
| Dose | 100 mg/kg | 200 mg/kg | 200:100 mg/kg |

1.5 h post-dose

| | | | |
|---|---|---|---|
| CSF | 40.4 μg/ml | 82.2 μg/ml | 2.03:1 |
| Blood | 58.5 μg/ml | 116.0 μg/ml | 1.98:1 |

4 h post-dose

| | | | |
|---|---|---|---|
| CSF | 11.0 μg/ml | 24.7 μg/ml | 2.25:1 |
| Blood | 15.6 μg/ml | 34.2 μg/ml | 2.19:1 |
| Brain | 22.6 μg/ml | 37.0 μg/ml | 1.63:1 |

CSF = cerebrospinal fluid.

There was an approximately proportional increase in the concentration of cG-2-AllylP in blood and CSF at 1.5 hours and in blood, CSF and brain at 4 hours following a single, oral dose. At 4 hours post-dose, the concentration of cG-2-AllylP in blood and brain tissue was approximately equivalent.

### Example 6: Effects of cG-2-AllylP in Mouse Model of Prader-Willi Syndrome

### A. Mouse Model C57BL/6-Magel2tm1Stw/J Jackson's Stock No: 009062

The mouse locus 7qB4/B5 (syntenic with the Prader-Willi region at chromosome position 15q11-q13 in humans) encompasses the cluster of paternally-expressed imprinted genes Magel2, Ndn, Mkrn3, and Peg12. As maternal imprinting silences the Magel2 allele, only the paternally inherited Magel2 allele is expressed. Heterozygous females were bred with wild-type males (or C57BL/6J inbred males) to maintain the line; as the resulting offspring will have no abnormal phenotype. To obtain “Magel2-null” offspring (both maternal and paternal Magel2 alleles are non-functional), wild-type females are bred with heterozygous males; allowing maternal transmission of the imprinted/silenced wild-type allele and paternal transmission of the Magel2-lacZ (null) allele. Magel2-null mice have disrupted circadian rhythm and metabolism, total activity, food consumption, weight before weaning, and fertility in both sexes, as well as increased adiposity after weaning and altered behavior. Magel2-null mice on this C57BL/6J genetic background recapitulate Prader-Willi syndrome. Because the genetic mutation in the Magel2-null animals is the same as the mutation in human beings, studies in Magel2-null mice are reasonably predictive of effects of compounds in human beings.

### B. Experimental Design

In vivo experiments were conducted in C57BL/6-Magel2tm1Stw/J (Magel2-null) mice at age P60 and their wild-type littermates during the light phase (littermates that were wild-type for Magel2 were used as controls). Ten mice per treatment group were used for studies 1 and 2, and six mice per group for study 3. In study 1: C57BL/6-Magel2tm1Stw/J (Magel2-null) mice were assessed on behavioral assays. Study 2 tested the efficacy of cG-2-AllylP in treating obesity and insulin levels in Magel2-null mice. Study 3 tested the IGF-1 level in Magel2-null mice. Because human beings have the same genetic abnormality, studies of Magel2-null mice are reasonably predictive of effects of compounds of Formulas I, II, III and IV in human beings with Prader-Willi Syndrome.

Experiments were conducted in line with the requirements of the UK Animals (Scientific Procedures) Act, 1986.

The mice were housed in plastic cages (35×30×12 cm), five in each and habituated to the animal facilities for at least a week before commencing the test. The room temperature (21° C.±2° C.), relative humidity (55%±5%), a 12-hour light-dark cycle (lights on 7 AM to 7 PM), and air exchange (16 times per h) were automatically controlled. The animals had free access to commercial food pellets and water.

Testing was performed during the light phase of the circadian cycle, with the order of testing being determined by the principle of conducting the most stressful tests last. Assays were designed to reproduce and expand on the original behavioral characterization of Magel2-null mice. Magel2-null and wild-type control mice were dosed once daily for 6 weeks prior to testing and tested 30 minutes following a dose of cG-2-AllylP (NNZ2591). Study groups and the treatment matrix are described in Table 2 below.

TABLE 2
Treatment matrix

| Group | | Dosing | |
|---|---|---|---|
| No. | Treatment Group | route | N |
| 1 | WT + Vehicle | Gavage | 10 |
| 2 | Magel2-null + Vehicle | Gavage | 10 |
| 3 | WT + NNZ2591 (100 mg/kg) | Gavage | 10 |
| 4 | Magel2-null + NNZ2591 (100 mg/kg) | Gavage | 10 |
| 5 | WT + NNZ2591 (200 mg/kg) | Gavage | 10 |
| 6 | Magel2-null + NNZ2591 (200 mg/kg) | Gavage | 10 |

WT = wild-type littermate control.

TransnetXY Automated Genotyping (www.transnetyx.com/). TRANSNETYX, INC., 8110 Cordova Rd. Suite 119, Cordova, TN 38016, USA, was used for genotyping.

All experiments were conducted with the experimenter blind to genotype and drug treatment. Separate investigators prepared and coded dosing solutions, allocated the mice to the study treatment groups, dosed the animals, and collected the behavioural data.

### Example 6a: Open Field Hypoactivity

To evaluate whether cG-2-AllylP is effective to treat the hypoactivity in PWS, Open Field studies were carried out. Magel2-null mice are less active in the Open Field than WT littermates, reflecting a decreased capacity for exercise.

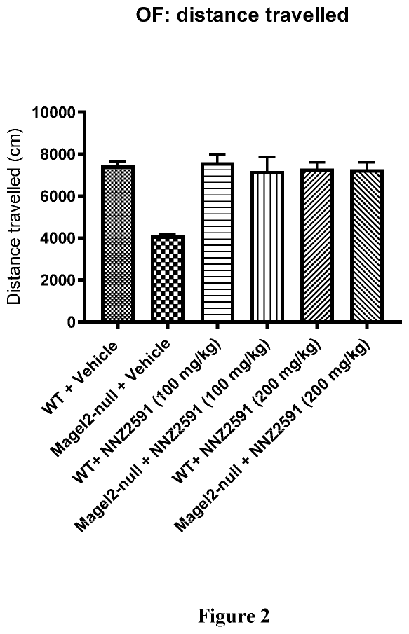

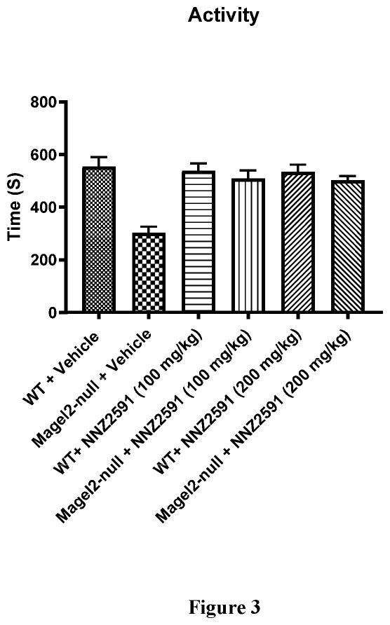

The results of experiments measuring distance travelled and time spent active are shown in FIG. 2 and FIG. 3, respectively.

The relative distance travelled or time spent active is shown on the vertical axis, and the mean of each treatment group is shown on the horizontal axis. Wild-type (WT) mice treated with vehicle alone (left column) were considered to travel 100% or to have 100% activity. Magel2-null mice treated with vehicle alone (second left column) showed significantly lower distance travelled and time spent active. All groups treated with cG-2-AllylP at either 100 mg/kg or 200 mg/kg were indistinguishable from the WT group treated with vehicle, indicating that either 100 mg/kg or 200 mg/kg normalized the mild to moderate hypoactivity in Magel2-null mice and had no effect on WT mice.

Open Field (Hypoactivity) - Distance travelled
ANOVA SUMMARY

| | |
|---|---|
| F | 123.3 |
| P value | <0.0001 |
| P value summary | **** |
| Significant diff. among means (P < 0.05)? | Yes |
| R Square | 0.9195 |

| Sidak's multiple comparison test | Summary | P Value |
|---|---|---|
| WT + Vehicle vs. Magel2-null + Vehicle | **** | <0.0001 |
| WT + Vehicle vs. WT + NNZ2591 (100 mg/kg) | ns | 0.9788 |
| WT + Vehicle vs. WT + NNZ2591 (200 mg/kg) | ns | 0.9809 |
| Magel2-null + Vehicle vs. Magel2-null + | **** | <0.0001 |
| NNZ2591 (100 mg/kg) | | |
| Magel2-null + Vehicle vs. Magel2-null + | **** | <0.0001 |
| NNZ2591 (200 mg/kg) | | |
| WT + NNZ2591 (100 mg/kg) vs. Magel2-null + | ns | 0.3635 |
| NNZ2591 (200 mg/kg) | | |
| WT + NNZ2591 (100 mg/kg) vs. Magel2-null + | ns | 0.1314 |
| NNZ2591 (100 mg/kg) | | |
| WT + NNZ2591 (200 mg/kg) vs. Magel2-null + | ns | >0.9999 |
| NNZ2591 (200 mg/kg) | | |

Open Field (Hypoactivity) - Motility
ANOVA summary

| | |
|---|---|
| F | 116.3 |
| P value | <0.0001 |
| P value summary | **** |
| Significant diff. among means (P < 0.05)? | Yes |
| R Square | 0.9150 |

| Sidak's multiple comparisons test | Summary | P Value |
|---|---|---|
| WT + Vehicle vs. WT + Magel2-null + Vehicle | **** | <0.0001 |
| WT + Vehicle vs. WT + NNZ2591 (100 mg/kg) | ns | 0.8254 |
| WT + Vehicle vs. WT + NNZ2591 (200 mg/kg) | ns | 0.5929 |
| Magel2-null + Vehicle vs. WT + NNZ2591 | **** | <0.0001 |
| (100 mg/kg) | | |
| Magel2-null + Vehicle vs. Magel2-null + | **** | <0.0001 |
| NNZ2591 (200 mg/kg) | | |
| Magel2-null + NNZ2591 (100 mg/kg) vs. | ns | 0.9993 |
| Magel2-null + NNZ2591 (200 mg/kg) | | |
| WT + NNZ2591 (100 mg/kg) vs. Magel2-null + | ns | 0.1754 |
| NNZ2591 (100 mg/kg) | | |
| WT + NNZ2591 (200 mg/kg) vs. Magel2-null + | ns | 0.1095 |
| NNZ2591 (200 mg/kg) | | |

ANOVA = analysis of variance;
ns = not significant;
WT = wildtype littermate control;
**** = p < 0.0001

### Example 6b: Nest Building

For small rodents, nests are important in heat conservation as well as reproduction and shelter. Nest building is an activity needed for mice to raise their offspring and is an indicator of social adaptation and activities of daily living. Typically, changes in nesting behaviors, such as failure to create a nest, indicate a change in health or welfare. Nesting behavior is sensitive to many genetic mutations underlying pathological disease states.

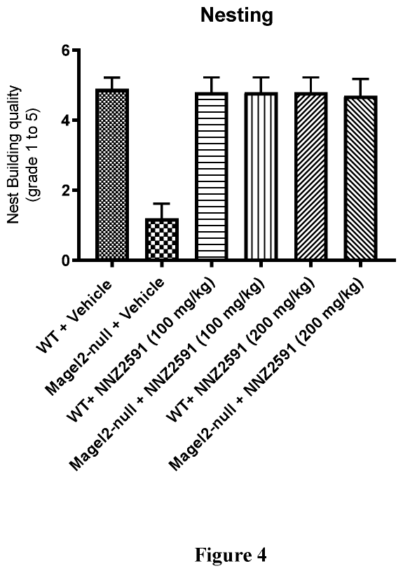

Nesting behavior is impaired in Magel2-null mice. Therefore, to determine if cG-2-AllylP might restore the quality of nest building, a series of studies were carried out. Results are shown in FIG. 4. The vertical axis shows nest building quality on a grade of 1-5, and the horizontal axis shows the mean of each treatment group.

Wild-type mice treated with vehicle exhibited nest building quality of about 5 (left column). In contrast, Magel2-null mice treated with vehicle only (second column from left) built nests of substantially lower quality. All groups treated with cG-2-AllylP at both 100 mg/kg and 200 mg/kg were indistinguishable from the WT group, indicating that either dose normalized the abnormal nest building in the Magel2-null mice and had no effect on WT mice.

Nesting
ANOVA summary

| | |
|---|---|
| F | 124.3 |
| P value | <0.0001 |
| P value summary | **** |
| Significant diff. among means (P < 0.05)? | Yes |
| R Square | 0.9201 |

| Sidak's multiple comparisons test | Summary | P Value |
|---|---|---|
| WT + Vehicle vs. Magel2-null + Vehicle | **** | <0.0001 |
| WT + Vehicle vs. WT + NNZ2591 (100 mg/kg) | ns | 0.9993 |
| WT + Vehicle vs. WT + NNZ2591 (200 mg/kg) | ns | 0.9993 |
| Magel2-null + Vehicle vs. Magel2-null + | **** | <0.0001 |
| NNZ 2591 (100 mg/kg) | | |
| Magel2-null + Vehicle vs. Magel2-null + | **** | <0.0001 |
| NNZ 2591 (200 mg/kg) | | |
| Magel2-null + NNZ2591 (100 mg/kg) vs. | ns | 0.9993 |
| Magel2-null + NNZ2591 (200 mg/kg) | | |
| WT + NNZ2591 (100 mg/kg) vs. Magel2-null + | ns | >0.9999 |
| NNZ 2591 (100 mg/kg) | | |
| WT + NNZ2591 (200 mg/kg) vs. Magel2-null + | ns | 0.9993 |
| NNZ2591 (200 mg/kg) | | |

ANOVA = analysis of variance;
ns = not significant;
WT = wildtype littermate control;
**** = p < 0.00001.

### Example 6c: Social Preference and Interaction

People with Prader-Willi Syndrome show deficits in social behavior compared with people without Prader-Willi Syndrome. Two tests were carried out to assess the social behavior deficits of Magel2-null mice.

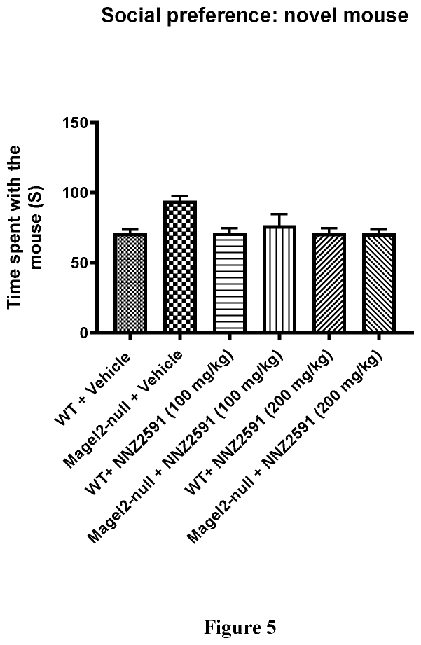

The partition test of social preference measures the preference shown for an object versus a familiar mouse. Results of the test are shown in FIG. 5. Magel2-null mice treated with vehicle alone (second left column) showed significantly more time showing preference for an object versus a familiar mouse than wild-type (WT) mice treated with vehicle alone (left column). All groups treated with cG-2-AllylP at either 100 mg/kg or 200 mg/kg were indistinguishable from the WT group treated with vehicle, indicating that either 100 mg/kg or 200 mg/kg normalized this social behavior deficit in Magel2-null mice and had no effect on WT mice.

Social preference
ANOVA Summary

| | |
|---|---|
| F | 44.93 |
| P value | <0.0001 |
| P value summary | **** |
| Significant diff. among means (P < 0.05)? | Yes |
| R Square | 0.8062 |

| Sidak's multiple comparisons test | Summary | P Value |
|---|---|---|
| WT + Vehicle vs. Magel2-null + Vehicle | **** | <0.0001 |
| WT + Vehicle vs. WT + NNZ2591 (100 mg/kg) | ns | >0.9999 |
| WT + Vehicle vs. WT + NNZ2591 (200 mg/kg) | ns | >0.9999 |
| Magel2-null + Vehicle vs. Magel2-null + | **** | <0.0001 |
| NNZ2591 (100 mg/kg) | | |
| Magel2-null + Vehicle vs. Magel2-null + | **** | <0.0001 |
| NNZ2591 (200 mg/kg) | | |
| WT + NNZ2591 (100 mg/kg) vs. Magel2-null + | ns | >0.9999 |
| NNZ2591 (200 mg/kg) | | |
| Magel2-null + NNZ2591 (100 mg/kg) vs. | * | 0.0380 |
| Magel2-null + NNZ2591 (200 mg/kg) | | |
| WT + NNZ2591 (100 mg/kg) vs. Magel2-null + | ns | 0.0975 |
| NNZ2591 (100 mg/kg) | | |
| WT + NNZ2591 (200 mg/kg) vs. Magel2-null + | ns | >0.9999 |
| NNZ2591 (200 mg/kg) | | |

ANOVA = analysis of variance;
ns = not significant;
WT = wildtype littermate control;
**** = p < 0.0001

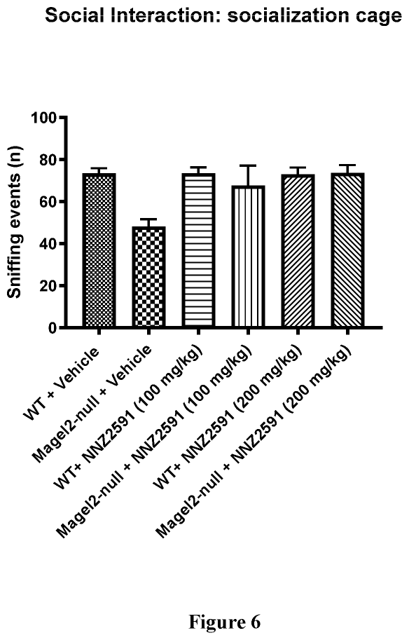

The sniffing test of social interaction measures the number of sniffing events. Results of the test are shown in FIG. 6. Magel2-null mice treated with vehicle alone (second left column) showed significantly fewer sniffing events than wild-type (WT) mice treated with vehicle alone (left column). All groups treated with cG-2-AllylP at either 100 mg/kg or 200 mg/kg were indistinguishable from the WT group treated with vehicle, indicating that either 100 mg/kg or 200 mg/kg normalized this social behavior deficit in Magel2-null mice and had no effect on WT mice.

Social interaction
ANOVA Summary

| | |
|---|---|
| F | 43.86 |
| P value | <0.0001 |
| P value summary | **** |
| Significant diff. among means (P < 0.05)? | Yes |
| R Square | 0.8024 |

| Sidak's multiple comparisons test | Summary | P Value |
|---|---|---|
| WT + Vehicle vs. Magel2-null + Vehicle | **** | <0.0001 |
| WT + Vehicle v. WT + NNZ2591 (100 mg/kg) | ns | >0.9999 |
| WT + Vehicle vs. WT + NNZ2591 (200 mg/kg) | ns | >0.9999 |
| Magel2-null + Vehicle vs. Magel2-null + NNZ2591 | **** | <0.0001 |
| (100 mg/kg) | | |
| Magel2-null + Vehicle vs. Magel2-null + NNZ2591 | **** | <0.0001 |
| (200 mg/kg) | | |
| Magel2-null + NNZ2591 (100 mg/kg) vs. | ns | 0.0665 |
| Magel2-null + NNZ2591 (200 mg/kg) | | |
| WT + NNZ2591 (100 mg/kg) vs. Magel2-null + | ns | 0.0749 |
| NNZ2591 (100 mg/kg) | | |
| WT + NNZ2591 (200 mg/kg) vs. Magel2-null + | ns | >0.9999 |
| NNZ2591 (200mg/kg) | | |

ANOVA = analysis of variance;
ns = not significant;
WT = wildtype littermate control;
**** = p < 0.0001

### Example 6d: Novel Object Recognition (NOR)

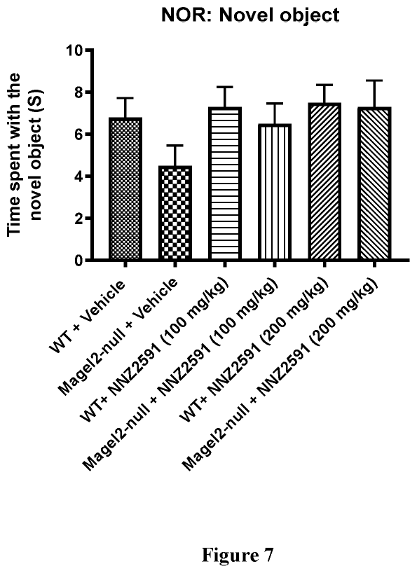

The NOR test is used to evaluate cognition, based on the tendency for mice to spend more time exploring a novel object that a familiar object. Magel2-null mice display a lack of discrimination between known and novel objects. The results of the NOR test are shown in FIG. 7. Magel2-null mice treated with vehicle alone (second left column) showed significantly less time spent with the novel object than wild-type (WT) mice treated with vehicle alone (left column). All groups treated with cG-2-AllylP at either 100 mg/kg or 200 mg/kg were indistinguishable from the WT group treated with vehicle, indicating that either 100 mg/kg or 200 mg/kg normalized this cognitive deficit in Magel2-null mice and had no effect on WT mice.

Novel object recognition
ANOVA Summary

| | |
|---|---|
| F | 12.63 |
| P value | <0.0001 |
| P value summary | **** |
| Significant diff. among means (P < 0.05)? | Yes |
| R Square | 0.5391 |

| Sidak's multiple comparisons test | Summary | P Value |
|---|---|---|
| WT + Vehicle vs. Magel2-null + Vehicle | **** | <0.0001 |
| WT + Vehicle vs. WT + NNZ2591 (100 mg/kg) | ns | 0.9152 |
| WT + Vehicle vs. WT + NNZ2591 (200 mg/kg) | ns | 0.6436 |
| Magel2-null + Vehicle vs. Magel2-null + | *** | 0.0003 |
| NNZ2591 (100 mg/kg) | | |
| Magel2-null + Vehicle vs. Magel2-null + | **** | <0.0001 |
| NNZ2591 (200 mg/kg) | | |
| Magel2-null + NNZ2591 (100 mg/kg) vs. | ns | 0.4749 |
| Magel2-null + NNZ2591 (200 mg/kg) | | |
| WT + NNZ2591 (100 mg/kg) vs. | ns | 0.4749 |
| Magel2-null + NNZ2591 (100 mg/kg) | | |
| WT + NNZ2591 (200 mg/kg) vs. | ns | 0.9998 |
| Magel2-null + NNZ2591 (200 mg/kg) | | |

ANOVA = analysis of variance;
ns = not significant;
WT = wildtype littermate control;
**** = p < 0.0001

### Example 6e: Elevated Plus Maze

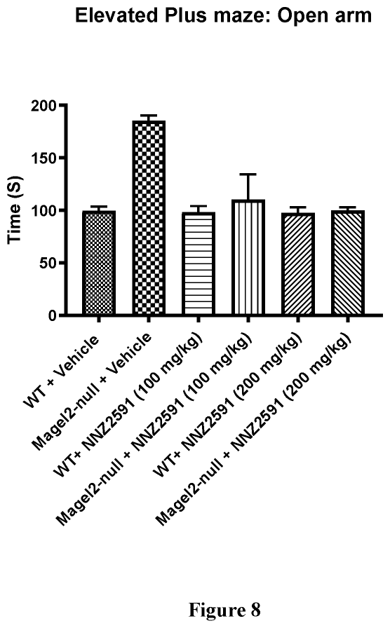

The Elevated Plus Maze test is used to assess anxiety related behaviors. Normal mice show a preference for being in closed arms over open arms. The time spent in open arms for each group is shown in FIG. 8. Magel2-null mice treated with vehicle alone (second left column, FIG. 8) showed significantly more time spent in open arms than wild-type (WT) mice treated with vehicle alone (left column). All groups treated with cG-2-AllylP at either 100 mg/kg or 200 mg/kg were indistinguishable from the WT group treated with vehicle, indicating that either 100 mg/kg or 200 mg/kg normalized this deficit in Magel2-null mice and had no effect on WT mice.

Elevated plus maze (time in open arm)
ANOVA Summary

| | |
|---|---|
| F | 106.5 |
| P value | <0.0001 |
| P value summary | **** |
| Significant diff. among means (P < 0.05)? | Yes |
| R Square | 0.908 |

| Sidak's multiple comparisons test | Summary | P Value |
|---|---|---|
| WT + Vehicle vs. Magel2-null + Vehicle | **** | <0.0001 |
| WT + Vehicle vs WT + NNZ2591 (100 mg/kg) | ns | >0.9999 |
| WT + Vehicle vs. WT + NNZ2591 (200 mg/kg) | ns | 0.9997 |
| Magel2-null + Vehicle vs. Magel2-null + | **** | <0.0001 |
| NNZ2591 (100 mg/kg) | | |
| Magel2-null + Vehicle vs. Magel2-null + | **** | <0.0001 |
| NNZ2591 (200 mg/kg) | | |
| WT + NNZ2591 (100 mg/kg) vs. Magel2-null + | ns | 0.0875 |
| NNZ2591 (100 mg/kg) | | |
| WT + NNZ2591 (200 mg/kg) vs. Magel2-null + | ns | 0.9991 |
| NNZ2591 (200 mg/kg) | | |

ANOVA = analysis of variance;
ns = not significant;
WT = wildtype littermate control;
**** = p < 0.0001

### Example 6f: Efficacy of cG-2-AllylP in Treating Obesity and Hormonal Abnormalities in Magel2-Null Mice

Prader-Willi Syndrome is associated with metabolic and hormonal abnormalities in humans. Magel2-null mice recapitulate some of the metabolic and hormonal aspects of humans with Prader-Willi Syndrome. Experiments were conducted to determine if cG-2-AllylP could have a normalizing effect in metabolic and hormonal aspects.

Whereas male Magel2-null mice exhibit a similar body weight gain curve from 6 to 18 weeks of age compared with their littermate wild-type controls, a significant increase in body weight is observed over time in female Magel2-null mice. Because female Magel2-null mice present a significant increase in body weight compared with their littermate wild-type controls, female Magel2-null mice were used for this study.

A. Effect of cG-2-AllylP on Fat Mass in Magel2-Null Mice

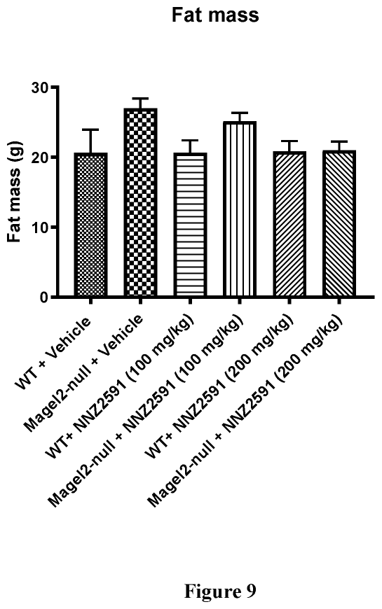

The mean fat mass of each treatment group was measured, as shown in FIG. 9. Magel2-null mice treated with vehicle alone (second left column) showed significantly higher fat mass than WT mice treated with vehicle alone (left column). Magel2-null mice treated with cG-2-AllylP (200 mg/kg) were indistinguishable from the WT group, indicating that this dose normalized the fat mass in the Magel2-null mice. There was no effect on WT mice treated with cG-2-AllylP.

Effect on fat mass
ANOVA Summary

| | |
|---|---|
| F | 13.49 |
| P value | <0.0001 |
| P value summary | **** |
| Significant diff. among means (P < 0.05)? | Yes |
| R Square | 0.6921 |

| Sidak's multiple comparisons test | Summary | P Value |
|---|---|---|
| WT + Vehicle vs. Magel2-null + Vehicle | **** | <0.0001 |
| WT + Vehicle vs. WT + NNZ2591 (100 mg/kg) | ns | >0.9999 |
| WT + Vehicle vs. WT + NNZ2591 (200 mg/kg) | ns | >0.9999 |
| Magel2-null + Vehicle vs. Magel2-null + | ns | 0.5655 |
| NNZ2591 (100 mg/kg) | | |
| Magel2-null + Vehicle vs. Magel2-null + | **** | <0.0001 |
| NNZ2591 (200 mg/kg) | | |
| Magel2-null + NNZ2591 (100 mg/kg) vs. | ** | 0.0044 |
| Magel2-null + NNZ2591 (200 mg/kg) | | |
| WT + NNZ2591 (100 mg/kg) vs. Magel2-null + | ** | 0.0019 |
| NNZ2591 (100 mg/kg) | | |
| WT + NNZ2591 (200 mg/kg) vs. Magel2-null + | ns | >0.9999 |
| NNZ2591 (200 mg/kg) | | |

ANOVA = analysis of variance;
ns = not significant;
WT = wildtype littermate control;
**** = p < 0.0001

B. Effect of cG-2-AllylP on Insulin Levels in Magel2-Null Mice

Insulin was measured for each group. The results are shown in Table 3 below. Magel2-null mice treated with vehicle alone showed significantly higher insulin levels than WT mice treated with vehicle alone. Magel2-null mice treated with cG-2-AllylP (200 mg/kg) were indistinguishable from the WT group, indicating that this dose normalized the insulin level in the Magel2-null mice. There was no effect on WT mice treated with cG-2-AllylP.

TABLE 3

| | | | WT + | Magel2- | WT + | Magel2-null + |
|---|---|---|---|---|---|---|
| | | | NNZ2591 | null + | NNZ2591 | NNZ2591 |
| | | Magel2- | 100 | NNZ2591 | 200 | 200 |
| | WT | null | mg/kg | 100 mg/kg | mg/kg | mg/kg |
| | (mean ± | (mean ± | (mean ± | (mean ± | (mean ± | (mean ± |
| Measurement | SEM) | SEM) | SEM) | SEM) | SEM) | SEM) |
| Insulin (pM) | 110 | 173 | 112 | 143 | 115 | 119 |
| | | (p < 0.005) | | (p < 0.005) | | (ns) |

ns = not significant;
WT = wildtype littermate control.

### Example 6g: IGF-1 Level in Magel2-Null mice

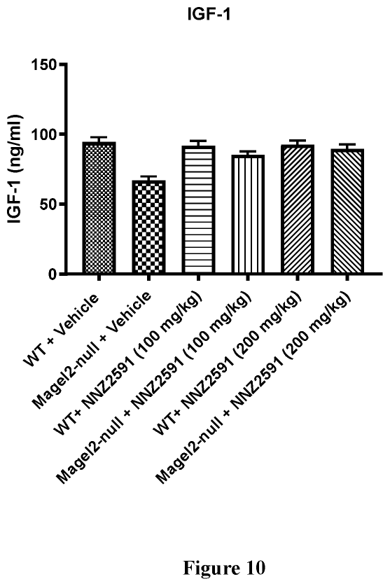

Prader-Willi Syndrome may be associated with altered IGF-1 levels in humans. The level of circulating IGF-1 was measured in each group, as shown in FIG. 10. Magel2-null mice treated with vehicle alone (second left column) showed significantly lower circulating IGF-1 than WT mice treated with vehicle alone (left column). Magel2-null mice treated with cG-2-AllylP (200 mg/kg) were indistinguishable from the WT group, indicating that this dose normalized the circulating IGF-1 levels in the Magel2-null mice. There was no effect on WT mice treated with cG-2-AllylP.

IGF-1 level
ANOVA Summary

| | |
|---|---|
| F | 66.7 |
| P value | 0.0001 |
| P value summary | **** |
| Significant diff. among means (P < 0.05)? | Yes |
| R Square | 0.9175 |

| Sidak's multiple comparisons test | Summary | P Value |
|---|---|---|
| WT + Vehicle vs. Magel2-null + Vehicle | **** | <0.0001 |
| WT + Vehicle vs. WT + NNZ2591 (100 mg/kg) | ns | 0.5987 |
| WT + Vehicle vs. WT + NNZ2591 (200 mg/kg) | ns | 0.8436 |
| WT + NNZ2591 (100 mg/kg) vs. Magel2-null + | ** | 0.0042 |
| NNZ2591 (100 mg/kg) | | |
| WT + NNZ2591 (200 mg/kg) vs. Magel2-null + | ns | 0.4665 |
| NNZ2591 (200 mg/kg) | | |
| Magel2-null + Vehicle vs. Magel2-null + | **** | <0.0001 |
| NNZ2591 (100 mg/kg) | | |

ANOVA = analysis of variance;
ns = not significant;
WT = wildtype littermate control;
**** = p < 0.0001

### Example 7: Effects of Cyclo-L-Glycyl-L-2-Allylproline in Human Beings with Prader-Willi Syndrome

### Methods

For the proposed study thirty subjects with Prader-Willi syndrome are recruited. Subjects are females and males aged between 4 and 20 years (Mean=12.1 SD=4.4). All subjects have confirmed loss of function in the 15q11-q13 region on the paternal copy of chromosome 15 and also meet diagnostic criteria for Prader-Willi syndrome based on assessment of behavioral and somatic symptoms. The majority of subjects (72%) are being treated with rhGH at screening. Subjects are instructed that concomitant medications are to be stable for at least six weeks prior to study.

The study is a randomized, double-blind placebo controlled parallel study with subjects randomized 1:1:1 to placebo, 6 mg/kg or 12 mg/kg of Cyclo-L-Glycyl-L-2-Allylproline is to be administered as an oral liquid twice daily for eighty-four days.

Subjects are tested at baseline using the following instruments: the Hyperphagia Questionnaire for Clinical Trials (HQ-CT), the Clinical Global Impression of Severity (CGI-S), the Caregiver Global Impression of Change (CaGI-I), the Aberrant Behavior Checklist (ABC) and ABC Subscales, the Social Responsiveness Scale, the Repetitive Behavior Scale-Revised (RBS-R), the PWS Anxiety and Distress Questionnaire (PADQ) and the Children's Yale-Brown Obsessive-Compulsive Scale (CY-BOCS). In addition, the following measurements are made at baseline: Body Fat Assessment (BFA), fasting blood glucose, blood lipids (HDL, LDL, VLDL, triglycerides), serum IGF-1 (total, bound to IGF-1 Binding Protein-3 [IGFBP-3]), fasting insulin and the Homeostatic Model Assessment for Insulin Resistance (HOMA-IR).

The effect of treatment with Cyclo-L-Glycyl-L-2-Allylproline is analyzed by conducting a repeated analysis of covariance (ANCOVA) on change from baseline scores for each of the measures plus the Clinical Global Impression of Change (CGI-I). Safety is assessed using caregiver reports of adverse events, a panel of standard laboratory tests and electrocardiograms (ECG).

### Indicative Results

There will be no difference in the incidence of treatment-emergent adverse events between placebo and either dose and no treatment-related pattern of abnormalities on laboratory tests or ECG measures. All adverse events are of short duration and mild severity. No Serious Adverse Events are reported. All subjects complete the study.

Analysis of efficacy would reveal the following:
Dose-dependent, statistically significant differences between active and placebo on improvement as measured by CGI-I.
Dose-dependent, statistically significant difference in change from baseline between active and placebo on Prader-Willi Distress Questionnaire (PADQ).
Statistically significant difference in change from baseline on CY-BOCS between active and placebo.
Dose-dependent, statistically significant difference in change from baseline between active and placebo on free IGF-1.
Trends toward reduction in fasting blood glucose and BFA between active and placebo.

1. A method of treating Prader-Willi Syndrome in a subject, comprising administering to the subject a therapeutically effective amount of a compound of Formulas I, II, III or IV, or a pharmaceutically acceptable salt, hydrate, stereoisomer, or prodrug thereof: 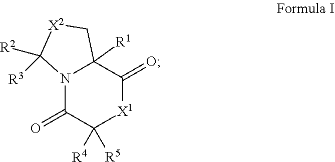
wherein:
X¹ is selected from the group consisting of NR′, O, and S;
X² is selected from the group consisting of CH₂, NR′, O, and S;
R¹, R², R³, R⁴, and R⁵ are independently selected from the group consisting of hydrogen, halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, alkynyl, 3-10-membered carbocycle, and 3-10-membered heterocycle,
wherein each alkyl, heteroalkyl, alkenyl, alkynyl, 3-10-membered carbocycle, and 3-10-membered heterocycle is unsubstituted or substituted with one or more substituents selected from the group consisting of halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, and alkynyl;
wherein each R′ is independently selected from the group consisting of hydrogen, alkyl, heteroalkyl, alkenyl, alkynyl, 3-10-membered carbocycle, and 3-10-membered heterocycle;
or R⁴ and R⁵ taken together are —CH₂—(CH₂)ₙ—CH₂— where n is an integer from 0-6;
or R² and R³ taken together are —CH₂—(CH₂)ₙ—CH₂— where n is an integer from 0-6;
with the proviso that when R¹ is CH₃, R² is hydrogen, R³ is hydrogen, and R⁴ is hydrogen, then R⁵ is not benzyl; and when R¹ is hydrogen, then at least one of R² and R³ is not hydrogen.

2. The method of claim 1, wherein R¹ is selected from the group consisting of hydrogen, —CH₃, and —CH₂CHCH₂.

3. The method of claim 1 or claim 2, wherein R² is selected from the group consisting of hydrogen and —CH₃.

4. The method of any one of claims 1 to 3, wherein R³ is selected from the group consisting of hydrogen and —CH₃.

5. The method of any one of claims 1 to 4, wherein X¹ is NH.

6. The method of any one of claims 1 to 5, wherein X² is selected from the group consisting of CH₂ and S.

7. The method of any one of claims 1 to 6, wherein R⁴ and R⁵ are each hydrogen, or taken together are selected from the group consisting of —CH₂—(CH₂)₃—CH₂— and —CH₂—(CH₂)₂—CH₂—.

8. The method of any one of claims 1 to 7, wherein the compound of Formula I is selected from the group consisting of:

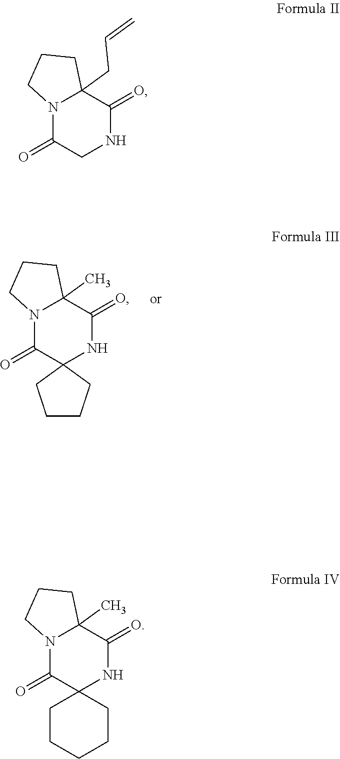 [SMILES: C=CCC12CCCN1C(=O)CNC2=O ; CC12CCCN1C(=O)C1(CCCC1)NC2=O ; CC12CCCN1C(=O)C1(CCCCC1)NC2=O]

9. The method of any one of claims 1 to 8, wherein the compound of Formula I is:

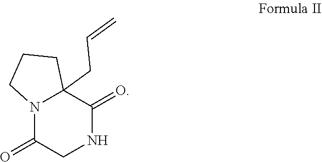

10. The method of any one of claims 1 to 9, wherein the treatment comprises preventing or reducing the likelihood or severity of one or more symptoms of Prader-Willi Syndrome.

11. The method of any one of claims 1 to 10, wherein Prader-Willi Syndrome is assessed using one or more clinical tests selected from the group consisting of genetic testing, IGF-1 in serum, Total IGF-1, Free IGF-1, Bound (to IGFBPs) IGF-1, IGF-1 in cerebral spinal fluid (CSF), Total IGF-1, Free IGF-1, IGFBPs in serum, IGFBP-1, -2, -3, -4, -5, -6 in serum, IGFBPs in CSF, IGFBP-1, -2, -3, -4, -5, -6 in CSF, blood glucose, blood lipids (HDL, LDL, VLDL, triglycerides), Homeostatic Model Assessment for Insulin Resistance (HOMA-IR), Body mass index (BMI), and Body fat assessment (BFA).

12. The method of claim 10 or claim 11, wherein the severity of the symptom is assessed using one or more clinical tests selected from the group consisting of the Hyperphagia Questionnaire for Clinical Trials (HQ-CT), the Clinical Global Impression of Severity (CGI-S), the Clinical Global Impression of Change (CGI-I), the Caregiver Global Impression of Change (CaGI-I), the Aberrant Behavior Checklist (ABC) and ABC Subscales, the Social Responsiveness Scale, the Repetitive Behavior Scale-Revised (RBS-R), the PWS Anxiety and Distress Questionnaire (PADQ), Intestinal microbiota composition (16S or other sequencing method), the Children's Yale-Brown Obsessive-Compulsive Scale (CY-BOCS), and the Montefiore Einstein Rigidity Scale-Revised-PWS (MERS-R-PWS).

13. The method of any one of claims 10 to 12, wherein the symptom or finding associated with Prader-Willi Syndrome is selected from the group consisting of early failure to thrive, excessive appetite (hyperphagia), obesity, type 2 diabetes, elevated blood insulin, metabolic syndrome, multiple endocrine abnormalities, hypotonia, hypogonadism, sleep disturbances, sleep apnoea, speech disorders, reduced pain sensitivity, poor bone health, strabismus, depigmentation, decreased gastrointestinal motility, scoliosis, adrenal insufficiencies, seizures, hypothyroidism, hypoglycaemia, hypogonadotropic hypogonadism, distinctive facial features, mild to moderate intellectual and learning disabilities, cognitive impairment, neurobehavioural disorders, intellectual disability, cognitive rigidity, heightened anxiety, severe temper outbursts, obsessive-compulsive behaviours, self-injurious behaviours, mental illness, autistic symptomatology, and growth hormone deficiency (GHD).

14. The method of any one of claims 1 to 13, wherein the compound of Formulas I, II, III, or IV is administered in combination with a therapeutic agent.

15. The method of claim 14, wherein the therapeutic agent is selected from the group consisting of recombinant human growth hormone (rhGH), human growth hormone, recombinant human IGF-1 (rhIGF-1), IGF-1, IGF-2, any IGF Binding Protein (IGFBP), IGFBP-3, insulin, any statin, any appetite suppressant, transforming growth factor-β1, activin, nerve growth factor, growth hormone binding protein, basic fibroblast growth factor, acidic fibroblast growth factor, the hst/Kfgk gene product, FGF-3, FGF-4, FGF-6, keratinocyte growth factor, androgen-induced growth factor, int-2, fibroblast growth factor, homologous factor-1 (FHF-1), FHF-2, FHF-3, FHF-4, keratinocyte growth factor 2, glial activating factor, FGF-10, FGF-16, ciliary neurotrophic factor, brain derived nerve growth factor, neurotrophin 3, neurotrophin 4, bone morphogenetic protein 2 (BMP-2), glial-cell line derived neurotrophic factor, activity-dependant neurotrophic factor, cytokine leukaemia inhibiting factor, oncostatin M, an interleukin, a-interferon, β-interferon, γ-interferon, consensus interferon, TNF-a, clomethiazole; kynurenic acid, Semax, tacrolimus, L-threo-1-phenyl-2-decanoylamino3, 3-morpholino-1-propanol, adrenocorticotropin-(4-9) analogue (ORG 2766), dizolcipine [MK-801], selegiline, NPS1506, GV1505260, MK-801, GV150526, 2,3-dihydroxy-6-nitro-7-sulfamoylbenzo(f)quinoxaline (NBQX), LY303070, LY300164, and the anti-MAdCAM-1 antibody MECA-367.

16. The method of claim 15, wherein the therapeutic agent is recombinant human growth hormone (rhGH).

17. The method of any one of claims 1 to 16, wherein the pharmaceutical composition is administered orally.

18. The method of any one of claims 1 to 17, wherein the compound of Formulas I, II, III or IV is administered in a dosage of from about 0.001 mg/kg to and including about 600 mg/kg.

19. The method of any one of claims 1 to 18, wherein the subject is a mammal.

20. The method of any one of claims 1 to 19, wherein the subject is a human.

21. The method of any one of claims 1 to 20, wherein the compound of Formulas I, II, III, or IV is administered in the form of a pharmaceutical composition comprising a pharmaceutically acceptable excipient.

22. The method of claim 21, wherein the pharmaceutically acceptable excipient is selected from the group consisting of binders, carriers, additives, adjuvants, microemulsions, coarse emulsions, and liquid crystals.

23. The method of claim 21 or claim 22, wherein the pharmaceutical composition is formulated as an oral solution, an oral suspension, or as a powder for preparing an oral solution or oral suspension.

24. The method of claim 21 or claim 22, wherein the pharmaceutical composition is formulated as a tablet or a capsule

25. Use of a compound of Formulas I, II, III or IV, or a pharmaceutically acceptable salt, hydrate, stereoisomer, or prodrug thereof: 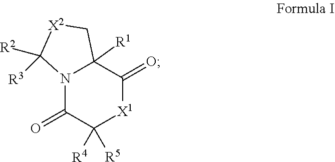
wherein:
X¹ is selected from the group consisting of NR′, O, and S;
X² is selected from the group consisting of CH₂, NR′, O, and S;
R¹, R², R³, R⁴, and R⁵ are independently selected from the group consisting of hydrogen, halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, alkynyl, 3-10-membered carbocycle, and 3-10-membered heterocycle,
wherein each alkyl, heteroalkyl, alkenyl, alkynyl, 3-10-membered carbocycle, and 3-10-membered heterocycle is unsubstituted or substituted with one or more substituents selected from the group consisting of halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, and alkynyl;
wherein each R′ is independently selected from the group consisting of hydrogen, alkyl, heteroalkyl, alkenyl, alkynyl, 3-10-membered carbocycle, and 3-10-membered heterocycle;
or R⁴ and R⁵ taken together are —CH₂—(CH₂)ₙ—CH₂— where n is an integer from 0-6;
or R² and R³ taken together are —CH₂—(CH₂)ₙ—CH₂— where n is an integer from 0-6;
with the proviso that when R¹ is CH₃, R² is hydrogen, R³ is hydrogen, and R⁴ is hydrogen, then R⁵ is not benzyl; and when R¹ is hydrogen, then at least one of R² and R³ is not hydrogen;
in the manufacture of a medicament for the treatment of Prader-Willi Syndrome.

26. A compound of Formulas I, II, III or IV, or a pharmaceutically acceptable salt, hydrate, stereoisomer, or prodrug thereof: 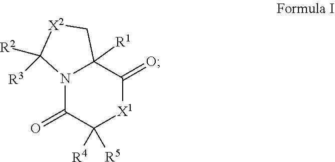
wherein:
X¹ is selected from the group consisting of NR′, O, and S;
X² is selected from the group consisting of CH₂, NR′, O, and S;
R¹, R², R³, R⁴, and R⁵ are independently selected from the group consisting of hydrogen, halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, alkynyl, 3-10-membered carbocycle, and 3-10-membered heterocycle,
wherein each alkyl, heteroalkyl, alkenyl, alkynyl, 3-10-membered carbocycle, and 3-10-membered heterocycle is unsubstituted or substituted with one or more substituents selected from the group consisting of halogen, —OR′, —SR′, —NR′R′, —NO₂, —CN, —C(O)R′, —C(O)OR′, —C(O)NR′R′, —C(NR′)NR′R′, alkyl, heteroalkyl, alkenyl, and alkynyl;
wherein each R′ is independently selected from the group consisting of hydrogen, alkyl, heteroalkyl, alkenyl, alkynyl, 3-10-membered carbocycle, and 3-10-membered heterocycle;
or R⁴ and R⁵ taken together are —CH₂—(CH₂)ₙ—CH₂— where n is an integer from 0-6;
or R² and R³ taken together are —CH₂—(CH₂)ₙ—CH₂— where n is an integer from 0-6;
with the proviso that when R¹ is CH₃, R² is hydrogen, R³ is hydrogen, and R⁴ is hydrogen, then R⁵ is not benzyl; and when R¹ is hydrogen, then at least one of R² and R³ is not hydrogen;
for use in treating Prader-Willi Syndrome.

27. Use of a compound of any of Formulas I, II, III or IV for treating a subject having Prader-Willi Syndrome as described herein.

28. The method of any of claims 1 to 24, wherein said subject is a human being.

29. The use of any of claims 25 to 27 wherein said subject is a human being.

30. The method of any of claims 1 to 24, wherein said compound is cG-2-AllylP (Formula II), or Cyclic Cyclopentyl-G-2-MeP (Formula III), or Cyclic Cyclohexyl-G-2-MeP (Formula IV.

31. The method of claim 30, wherein said compound is formulated in an aqueous solution.

32. The use of any of claims 25 to 27, wherein said compound is cG-2-AllylP (Formula II), or Cyclic Cyclopentyl-G-2-MeP (Formula III), or Cyclic Cyclohexyl-G-2-MeP (Formula IV).

33. The use of claim 32, wherein said compound is formulated in an aqueous solution.

34. The method of any of claims 1 to 24, wherein the dose of said compound is from about 0.01 mg per kg of body weight (mg/kg) to including about 1000 mg/kg, alternatively from about 0.1 mg/kg to including about 500 mg/kg., or about 0.1 mg/kg. to and including about 200 mg/kg, or about 0.01, or 0.1, or 1, or 10, or 20, or 50, or 75, or 100, or 500, or 1000, or 5000 mg/kg, respectively.
